(19) United States
(12) Patent Application Publication
(10) Pub. No.: US 2024/0042205 A1
(43) Pub. Date: Feb. 8, 2024

(54) ANTENNA ARRANGEMENTS

(71) Applicant: COCHLEAR LIMITED, Macquarie University, NSW (AU)

(72) Inventors: Werner MESKENS, Macquarie University (AU); Wim BERVOETS, Macquarie University (AU); Martin Joseph SVEHLA, Macquarie University (AU)

(21) Appl. No.: 18/266,671

(22) PCT Filed: Dec. 10, 2021
(86) PCT No.: PCT/IB2021/061587

§ 371 Date: Jun. 12, 2023

Related U.S. Application Data
(60) Provisional application No. 63/123,734, filed on Dec. 10, 2020.

(51) Int. Cl. A61N 1/36; A61N 1/375

(52) U.S. Cl. CPC A61N 1/36038; A61N 1/375

(57) ABSTRACT

A system for magnetic induction communication between an implantable component and an external component, including an implantable component, the implantable component including magnetic induction radio communication circuitry connected to an implantable antenna arrangement of the implantable component, the implantable antenna arrangement comprising at least two coil antennas for radio communication and an external component including magnetic induction radio circuitry connected to a coil antenna of the external component, wherein the system is configured so that when the implantable antenna arrangement of the implantable component is implanted between a skull and skin of a human and the external component is worn on the head of the component during normal use the magnetic induction communication link between the external and the implantable component is active and effectively operating.

### CROSS-REFERENCE TO RELATED APPLICATIONS

This application claims priority to U.S. Provisional Application No. 63/123,734, entitled ANTENNA ARRANGEMENTS, filed on Dec. 10, 2020, naming Werner MESKENS of Mechelen, Belgium as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a system for magnetic induction communication between an implantable component and an external component, comprising an implantable component, the implantable component including magnetic induction radio communication circuitry connected to an implantable antenna arrangement of the implantable component, the implantable antenna arrangement comprising at least two coil antennas for radio communication, and an external component including magnetic induction radio circuitry connected to a coil antenna of the external component, wherein the system is configured so that when the implantable antenna arrangement of the implantable component is implanted between a skull and skin of a human and the external component is worn on the head of the component during normal use the magnetic induction communication link between the external and the implantable component is active and effectively operating.

In another exemplary embodiment, there is a device, comprising an implantable hermetically sealed biocompatible housing, electronics located inside the housing, a first implantable antenna coil, the first implantable antenna coil electrically connected to the electronics and/or the device is configured to connect the first implantable antenna coil to the electronics, and a second implantable antenna coil, the second implantable antenna coil electrically connected to the electronics and/or the device is configured to connect the second implantable antenna coil to the electronics, wherein the electronics includes magnetic induction radio communication circuitry, the device is configured to enable a magnetic induction communication link, and the device is implantable in a human recipient between skin and skull bone of the human.

In another exemplary embodiment, there is method, comprising establishing a first transcutaneous link for power transfer using magnetic induction between a first set of closely coupled coil antennas with a clearance less than 20 mm, an establishing a second transcutaneous link for data communication using magnetic induction between a second set of loosely coupled coil antennas with a clearance greater than 10 mm, wherein the first and second transcutaneous links are established by the same or respective different external components of a prosthetic hearing implant system and the same implanted component of the prosthetic hearing implant implanted in a human recipient, and at least one of (1) the second transcutaneous link is established using antenna diversity via the implantable component, or (2) the second transcutaneous link is established using antenna and receiver diversity by the implantable component.

In another exemplary embodiment, there is a method, comprising establishing a transcutaneous or subcutaneous data communication link using magnetic induction with an implanted component including magnetic induction radio communication circuitry connected to an implantable antenna arrangement of the implantable component comprising at least two coil antennas, wherein the method includes selecting one coil antenna of the at least two coil antennas for connection to the magnetic induction radio communication circuitry based on data based on a link quality associated with the selected one coil antenna, which selected antenna is used to establish the communication link.

In another exemplary embodiment, there is a method, comprising establishing a first transcutaneous link that is a power induction transfer using a first set of antennas, and establishing a second transcutaneous link for data transfer using a second set of antennas, wherein the first and second transcutaneous links are established by the same or respective different external components of a prosthetic hearing implant system and the same implanted component of the prosthetic hearing implant implanted in a human recipient, and the second link utilizes induction to communicate the data, wherein the prosthetic hearing implant implanted in the human recipient includes at least two coil antennas of which an antenna of the first set and an antenna of the second set are apart.

In an exemplary embodiment, there is a method, wherein the method is executed for an antenna arrangement of an implanted component of a prosthetic hearing implant implanted in a human recipient, the implanted component comprising at least two coil antennas, the method including establishing a first transcutaneous data link between an antenna arrangement of a first external component and an antenna arrangement of the implanted component, wherein the antenna arrangement of the first external component is close to or in an ear canal of the recipient, the first external component being a non-implantable component, and a second transcutaneous data link between an antenna arrangement of a second external component and the antenna arrangement of implanted component, wherein the antenna arrangement of the second external component is close to or in the ear canal of the recipient, the second implantable component being a non-implantable component.

In an exemplary embodiment, there is a method, wherein the method is executed for an antenna arrangement of an implanted component of a prosthetic hearing implant implanted in a human recipient, the implanted component comprising at least two coil antennas, the method including establishing a first transcutaneous data link between an antenna arrangement of a first external component and an antenna arrangement of the implanted component, wherein the antenna arrangement of the first external component is close to or in an ear canal of the recipient, the first external component being a non-implantable component, and a second data link between the first external component and a second external component, wherein an antenna arrangement of the second external component is close to or in the ear canal of the recipient, the second implantable component being a non-implantable component.

In an exemplary embodiment, there is a method, comprising implanting, on a right side of a first human, a first implantable component of a hearing prosthesis including a wide diameter antenna so that the wide diameter antenna is implanted behind or above a pinna of the first human, and implanting, on a right side of a second human, a second implantable component of a hearing prosthesis including a wide diameter antenna so that the wide diameter antenna is implanted behind or above a pinna of the second human, wherein the first and second implantable components are the same design, the orientations of the implantable components after implantation are substantially different, the design of the implantable components is a design that receives power via the wide diameter antenna and data via an antenna system separate from that associated with the wide diameter antenna including at least two antennas spaced away from the wide diameter antenna, and a link quality between the antenna system of the first implantable component and an antenna of an external component within a housing of a spine of a BTE device will be effective to communicate data to the antenna system of the first implant so that an effective hearing percept can be evoked in the first human, and a link quality between the antenna system of the second implantable component and the antenna of the external component within the housing of the spine of the BTE device will be effective to communicate data to the antenna system of the second implant so that an effective hearing percept can be evoked in the second human when the external component is worn by the second human in the same way as worn by the first human, all other things being equal.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIG. 1B is a top view of an exemplary hearing prosthesis in which at least some of the teachings detailed herein are applicable;

FIG. 1C is a side view of an exemplary hearing prosthesis in which at least some of the teachings detailed herein are applicable;

FIG. 1D is a view of an exemplary sight prosthesis in which at least some of the teachings herein are applicable;

FIG. 1E presents an exemplary external component that provides a baseline for an exemplary external component that is utilized with the teachings herein;

FIGS. 2A-B are exemplary functional block diagrams of a prosthesis that provides a baseline for the inventive teachings herein;

FIG. 2C presents an exemplary external component that provides a baseline for an exemplary external component that is utilized with the teachings herein;

FIGS. 3A-3C are exemplary functional block diagrams of cochlear implants that provides a baseline for the inventive teachings herein;

FIG. 4A is a simplified schematic diagram of a transceiver unit of an external device that provides a baseline for the inventive teachings herein;

FIG. 4B is a simplified schematic diagram of a transmitter unit of an external device that provides a baseline for the inventive teachings herein;

FIG. 4C is a simplified schematic diagram of a stimulator/receiver unit including a data receiver of an implantable device that provides a baseline for the inventive teachings herein;

FIG. 4D is a simplified schematic diagram of a stimulator/receiver unit including a data transceiver of an implantable device that provides a baseline for the inventive teachings herein;

FIG. 4E is a simplified schematic diagram of a stimulator/receiver unit including a data receiver and a communication component configured to vary the effective coil area of an implantable device that provides a baseline for the inventive teachings herein;

FIG. 4F is a simplified schematic diagram of a stimulator/receiver unit including a data transceiver and a communication component that provides a baseline for the inventive teachings herein;

FIGS. 5 and 6 and 7 present an exemplary implantable component that provides a baseline for an exemplary external component that is utilized with the teachings herein;

FIGS. 8 to 9D and 13 present exemplary external components that are exemplary external components utilized with the inventive implantable components herein;

FIGS. 10-12 and 14-24 present exemplary implantable components according to inventive embodiments herein;

FIGS. 25 and 26 present exemplary systems according to inventive systems herein;

FIGS. 27 and 29 and 31 present schematics showing spatial features that are applicable to at least some embodiments;

FIG. 28 presents an exemplary flowchart for an exemplary inventive method; and

FIG. 30 depicts an X-ray result of a human with an implant and an external component.

### DETAILED DESCRIPTION

Exemplary embodiments will be described in terms of a cochlear implant. That said, it is noted that the teachings detailed herein and/or variations thereof can be utilized with other types of hearing prostheses, such as by way of example, bone conduction devices, DACI/DACS/middle ear implants, etc. Indeed, any disclosure herein of an electrode array corresponds to an alternate disclosure of an actuator of a middle ear implant or a bone conduction device or a DACS/DACI, etc., and a disclosure of the alternate electronics of the implant to implement such. Still further, it is noted that the teachings detailed herein and/or variations thereof can be utilized with other types of prostheses, such as pacemakers, muscle stimulators, etc. In some instances, the teachings detailed herein and/or variations thereof are applicable to any type of implanted component that utilizes feedthroughs.

To be clear, the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

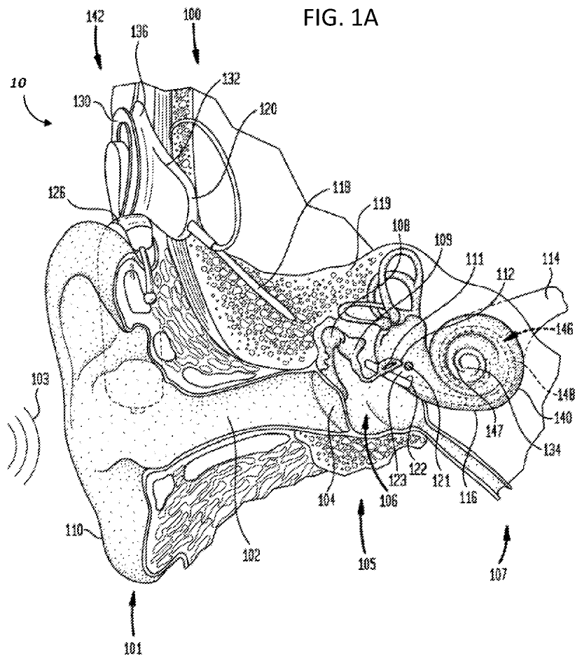

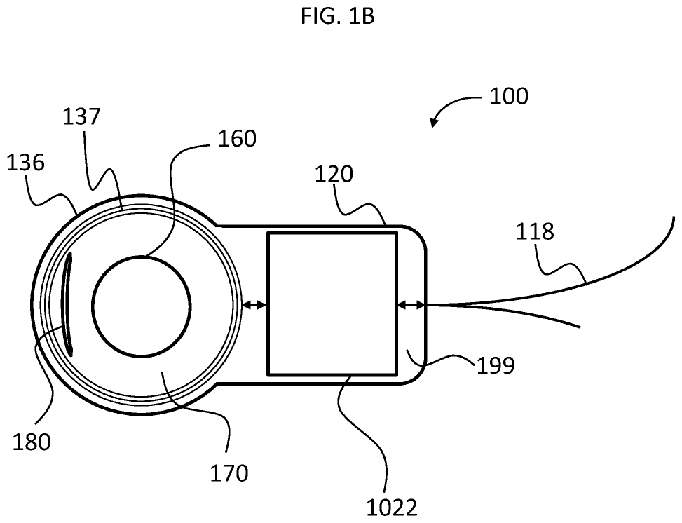

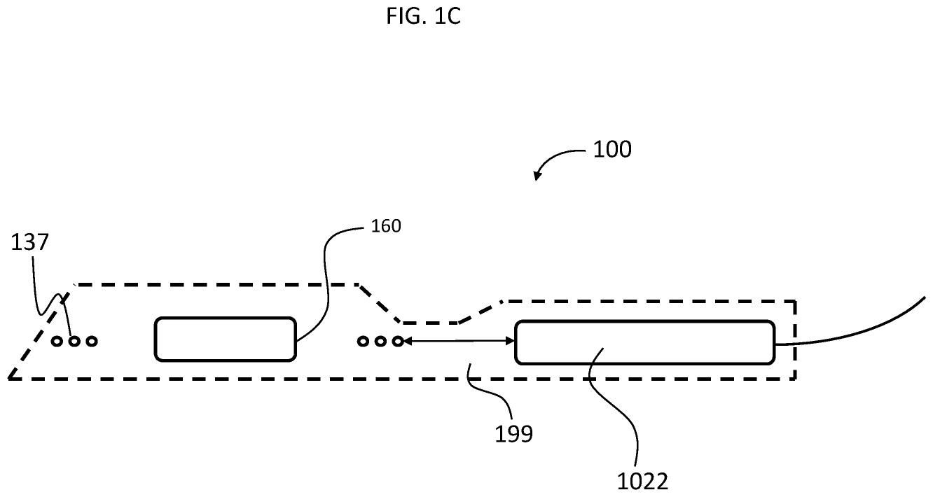

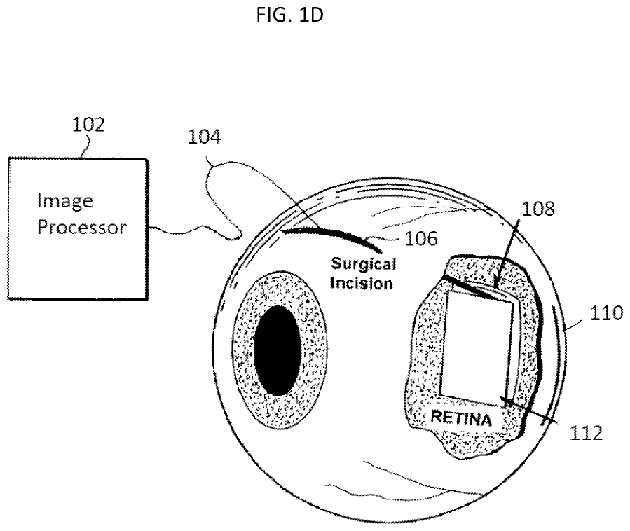

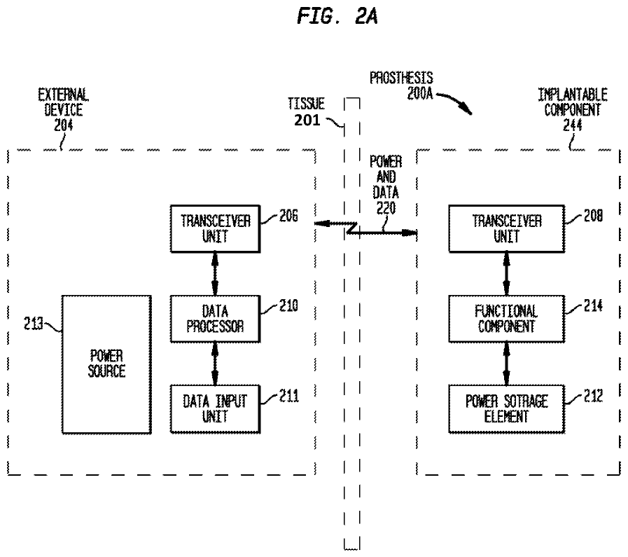

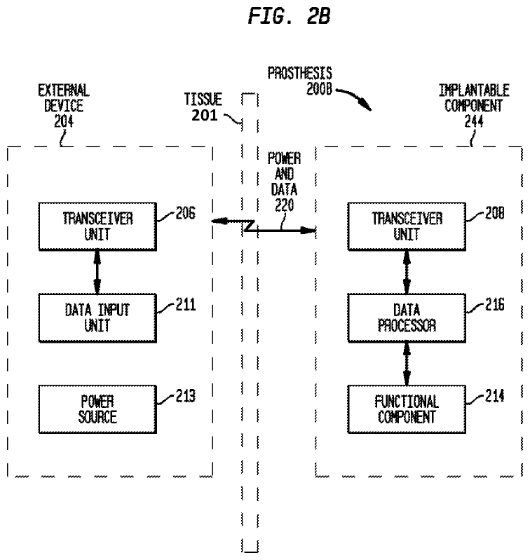

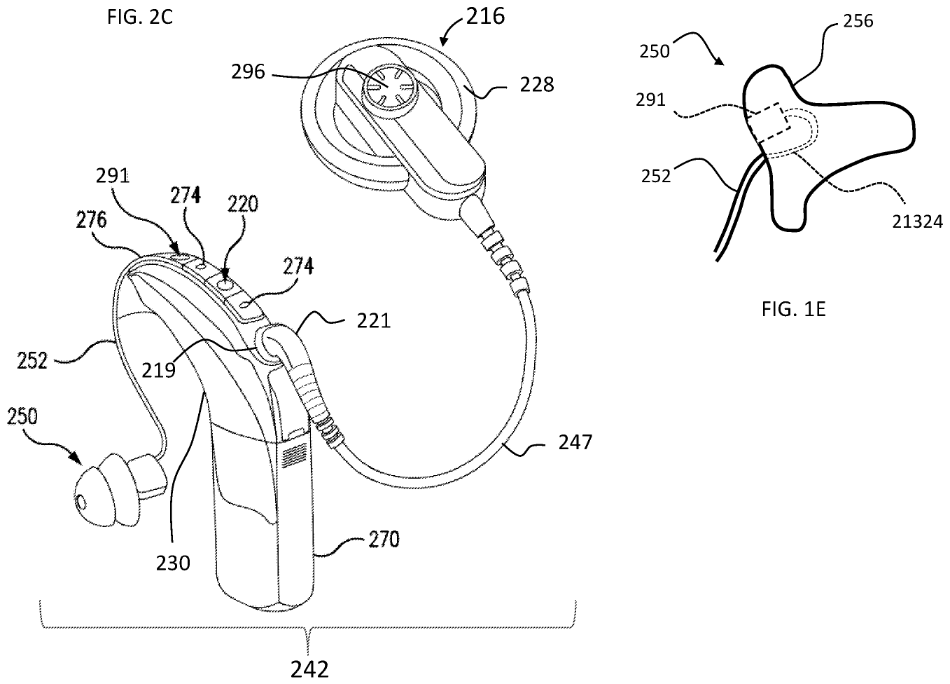

FIG. 1A is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. It is noted that the teachings detailed herein are applicable, in at least some embodiments, to partially implantable and/or totally implantable cochlear implants (i.e., with regard to the latter, such as those having an implanted microphone). It is further noted that the teachings detailed herein are also applicable to other stimulating devices that utilize an electrical current beyond cochlear implants (e.g., auditory brain stimulators, pacemakers, etc.). Additionally, it is noted that the teachings detailed herein are also applicable to other types of hearing prostheses, such as by way of example only and not by way of limitation, bone conduction devices, direct acoustic cochlear stimulators, middle ear implants, etc. Indeed, it is noted that the teachings detailed herein are also applicable to so-called hybrid devices. In an exemplary embodiment, these hybrid devices apply both electrical stimulation and acoustic stimulation to the recipient. Any type of hearing prosthesis to which the teachings detailed herein and/or variations thereof that can have utility can be used in some embodiments of the teachings detailed herein.

In view of the above, it is to be understood that at least some embodiments detailed herein and/or variations thereof are directed towards a body-worn sensory supplement medical device (e.g., the hearing prosthesis of FIG. 1A, which supplements the hearing sense, even in instances where all natural hearing capabilities have been lost). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities remaining and to recipients having no natural vision capabilities remaining). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant, and where the implanted cochlear implant includes a battery, that is recharged by the power provided from the external device 142.

In the illustrative arrangement of FIG. 1A, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1A, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with the teachings herein.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil assembly 137. Internal coil assembly 137 typically includes a wire antenna coil comprised of multiple turns of electrically insulated single-strand or multi-strand platinum or gold wire, as will be described in greater detail below.

Cochlear implant 100 further comprises a main implantable component 120 and an elongate electrode assembly 118. Collectively, the coil assembly 137, the main implantable component 120, and the electrode assembly 118 correspond to the implantable component of the system 10.

In some embodiments, internal energy transfer assembly 132 and main implantable component 120 are hermetically sealed within a biocompatible housing or within the device in general (the housing per se may not be hermetically sealed). In some embodiments, main implantable component 120 includes an implantable microphone assembly (not shown) and a sound processing unit (not shown) to convert the sound signals received by the implantable microphone or via internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown in FIG. 1A) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123, or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

FIG. 1B depicts an exemplary high-level diagram of the implantable component 100 of the system 10, looking downward from outside the skull towards the skull. As can be seen, implantable component 100 includes a magnet 160 that is surrounded by a coil 137 that is in two-way communication (although in some instances, the communication is one-way) with a receiver stimulator unit 1022, which in turn is in communication with the electrode assembly 118.

Still with reference to FIG. 1B, it is noted that the receiver stimulator unit 1022, and the magnet apparatus 160 are located in a housing made of an elastomeric material 199, such as by way of example only and not by way of limitation, silicone. Hereinafter, the elastomeric material 199 of the housing will be often referred to as silicone. However, it is noted that any reference to silicone herein also corresponds to a reference to any other type of component that will enable the teachings detailed herein and/or variations thereof, such as, by way of example and not by way of limitation only, bio-compatible rubber, etc.

As can be seen in FIG. 1B, the housing made of elastomeric material 199 includes a slit 180 (not shown in FIG. 1C, as, in some instances, the slit is not utilized). In some variations, the slit 180 has utilitarian value in that it can enable insertion and/or removal of the magnet apparatus 160 from the housing made of elastomeric material 199.

It is noted that magnet apparatus 160 is presented in a conceptual manner. In this regard, it is noted that in at least some instances, the magnet apparatus 160 is an assembly that includes a magnet surrounded by a biocompatible coating. Still further by way of example, magnet apparatus 160 is an assembly where the magnet is located within a container having interior dimensions generally corresponding to the exterior dimensions of the magnet. This container can be hermetically sealed, thus isolating the magnet in the container from body fluids of the recipient that penetrate the housing (the same principle of operation occurs with respect to the aforementioned coated magnet). In an exemplary embodiment, this container permits the magnet to revolve or otherwise move relative to the container. Additional details of the container will be described below. In this regard, it is noted that while sometimes the term magnet is used as shorthand for the phrase magnet apparatus, and thus any disclosure herein with respect to a magnet also corresponds to a disclosure of a magnet apparatus according to the aforementioned embodiments and/or variations thereof and/or any other configuration that can have utilitarian value according to the teachings detailed herein.

Briefly, it is noted that there is utilitarian value with respect to enabling the magnet to revolve within the container or otherwise move. In this regard, in an exemplary embodiment, when the magnet is introduced to an external magnetic field, such as in an Mill machine, the magnet can revolve or otherwise move to substantially align with the external magnetic field. In an exemplary embodiment, this alignment can reduce or otherwise eliminate the torque on the magnet, thus reducing discomfort and/or reducing the likelihood that the implantable component will be moved during the Mill procedure (potentially requiring surgery to place the implantable component at its intended location) and thus reduce and/or eliminate the demagnetization of the magnet.

Element 136 can be considered a housing of the coil, in that it is part of the housing 199.

With reference now to FIG. 1C, it is noted that the outlines of the housing made from elastomeric material 199 are presented in dashed line format for ease of discussion. In an exemplary embodiment, silicone or some other elastomeric material fills the interior within the dashed line, other than the other components of the implantable device (e.g., plates, magnet, stimulator, etc.). That said, in an alternative embodiment, silicone or some other elastomeric material substantially fills the interior within the dashed lines other than the components of the implantable device (e.g., there can be pockets within the dashed line in which no components and no silicone are located).

It is noted that FIGS. 1B and 1C are conceptual FIGs. presented for purposes of discussion. Commercial embodiments corresponding to these FIGs. can be different from that depicted in the figures.

FIG. 1D presents an exemplary embodiment of a neural prosthesis in general, and a retinal prosthesis and an environment of use thereof, in particular. In some embodiments of a retinal prosthesis, a retinal prosthesis sensor-stimulator 108 is positioned proximate the retina 110. In an exemplary embodiment, photons entering the eye are absorbed by a microelectronic array of the sensor-stimulator 108 that is hybridized to a glass piece 112 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 102 is in signal communication with the sensor-stimulator 108 via cable 104 which extends through surgical incision 106 through the eye wall (although in other embodiments, the image processor 102 is in wireless communication with the sensor-stimulator 108). In an exemplary embodiment, the image processor 102 is analogous to the sound processor/signal processors of the auditory prostheses detailed herein, and in this regard, any disclosure of the latter herein corresponds to a disclosure of the former in an alternate embodiment. The image processor 102 processes the input into the sensor-stimulator 108, and provides control signals back to the sensor-stimulator 108 so the device can provide processed and output to the optic nerve. That said, in an alternate embodiment, the processing is executed by a component proximate to or integrated with the sensor-stimulator 108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 108 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner. Indeed, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

Returning back to the cochlear implant embodiment, FIG. 2A is a baseline functional block diagram of a prosthesis 200A that presents basic features that are utilized. Prosthesis 200A comprises an implantable component 244 configured to be implanted beneath a recipient's skin or other tissue 201 and an external device 204. For example, implantable component 244 may be implantable component 100 of FIG. 1A, and external device may be the external device 142 of FIG. 1A. Similar to the embodiments described above with reference to FIG. 1A, implantable component 244 comprises a transceiver unit 208 which receives data and power from external device 204. External device 204 transmits power and data 220 via transceiver unit 206 to transceiver unit 208 via a magnetic induction data link 220. As used herein, the term receiver refers to any device or component configured to receive power and/or data such as the receiving portion of a transceiver or a separate component for receiving. The details of transmission of power and data to transceiver unit 208 are provided below. With regard to transceivers, it is noted at this time that while embodiments may utilize transceivers, separate receivers and/or transmitters may be utilized as appropriate. Herein, any disclosure of one corresponds to a disclosure of the other and vice versa.

Implantable component 244 may comprises a power storage element 212 and a functional component 214. Power storage element 212 is configured to store power received by transceiver unit 208, and to distribute power, as needed, to the elements of implantable component 244. Power storage element 212 may comprise, for example, a rechargeable battery 212. An example of a functional component may be a stimulator unit 120 as shown in FIG. 1B.

In certain embodiments, implantable component 244 may comprise a single unit having all components of the implantable component 244 disposed in a common housing. In other embodiments, implantable component 244 comprises a combination of several separate units communicating via wire or wireless connections. For example, power storage element 212 may be a separate unit enclosed in a hermetically sealed device, such as the housing, or the combination of the housing and other components, etc. The implantable magnet apparatus and plates associated therewith may be attached to or otherwise be a part of any of these units, and more than one of these units can include the magnet apparatus and plates according to the teachings detailed herein and/or variations thereof.

In the embodiment depicted in FIG. 2A, external device 204 includes a data processor 210 that receives data from data input unit 211 and processes the received data. The processed data from data processor 210 is transmitted by transceiver unit 206 to transceiver unit 208. In an exemplary embodiment, data processor 210 may be a sound processor, such as the sound processor of FIG. 1A for the cochlear implant thereof, and data input unit 211 may be a microphone of the external device.

FIG. 2B presents an alternate embodiment of the prosthesis 200A of FIG. 2A, identified in FIG. 2B as prosthesis 200B. As may be seen from comparing FIG. 2A to FIG. 2B, the data processor can be located in the external device 204 or can be located in the implantable component 244. In some embodiments, both the external device 204 and the implantable component 244 can include a data processor.

As shown in FIGS. 2A and 2B, external device 204 can include a power source 213. Power from power source 213 can be transmitted by transceiver unit 206 to transceiver unit 208 to provide power to the implantable component 244, as will be described in more detail below.

While not shown in FIGS. 2A and 2B, external device 204 and/or implantable component 244 include respective inductive communication components. These inductive communication components can be connected to transceiver unit 206 and transceiver unit 208, permitting power and data 220 to be transferred between the two units via magnetic induction.

As used herein, an inductive communication component includes both standard induction coils and inductive communication components configured to vary their effective coil areas.

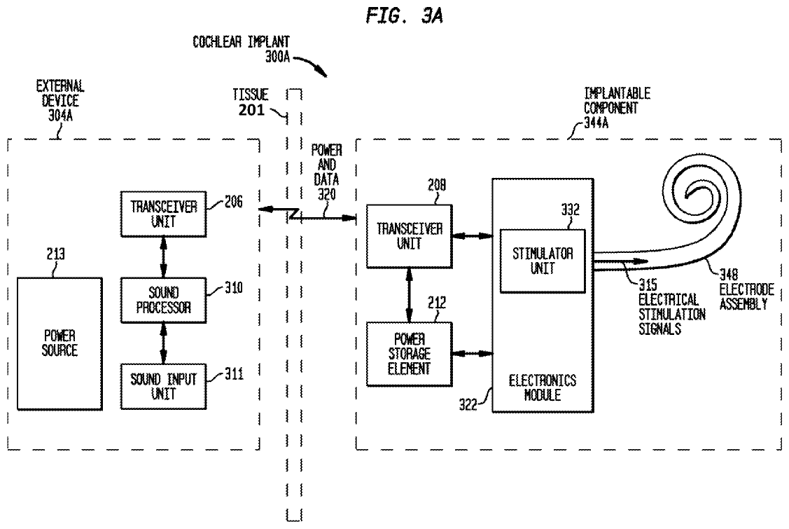

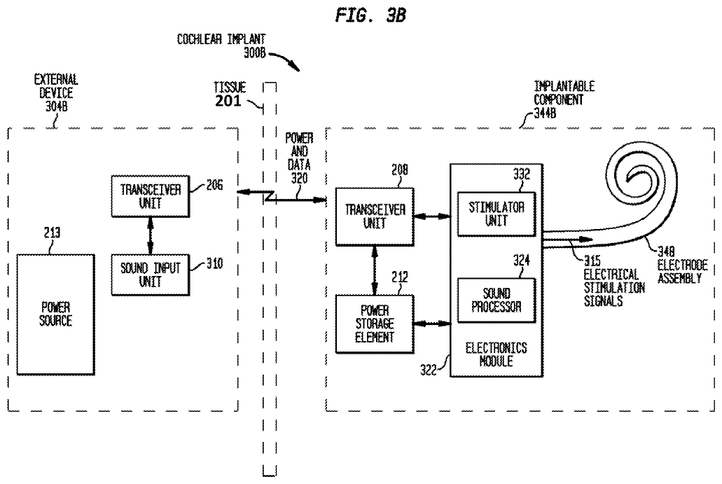

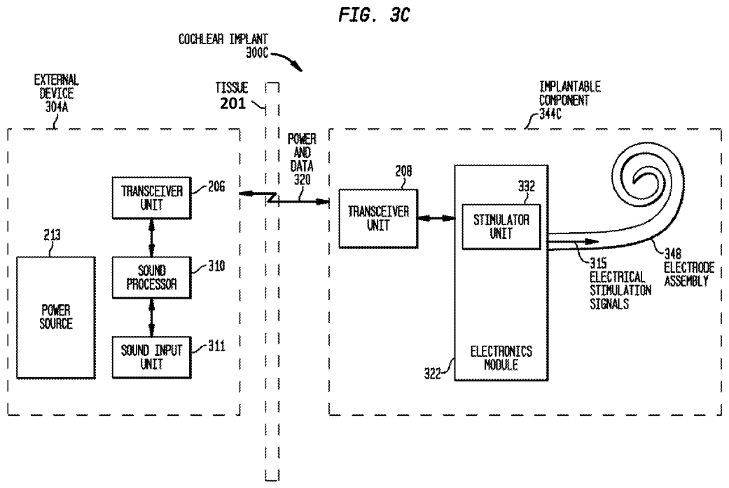

As noted above, prosthesis 200A of FIG. 2A may be a cochlear implant. In this regard, FIG. 3A provides additional details of an embodiment of FIG. 2A where prosthesis 200A is a cochlear implant. Specifically, FIG. 3A is a functional block diagram of a cochlear implant 300.

It is noted that the components detailed in FIGS. 2A and 2B may be identical to the components detailed in FIG. 3A, and the components of 3A may be used in the embodiments depicted in FIGS. 2A and 2B.

Cochlear implant 300A comprises an implantable component 344A (e.g., implantable component 100 of FIG. 1) configured to be implanted beneath a recipient's skin or other tissue 250, and an external device 304A. External device 304A may be an external component such as external component 142 of FIG. 1.

Similar to the embodiments described above with reference to FIGS. 2A and 2B, implantable component 344A comprises a transceiver unit 208 (which may be the same transceiver unit used in FIGS. 2A and 2B) which receives data and power from external device 304A. External device 304A transmits data and/or power 320 to transceiver unit 208 via a magnetic induction data link. This can be done while charging module 212.

Implantable component 344A also comprises a power storage element 212, electronics module 322 (which may include components such as sound processor 126 and/or may include a receiver stimulator unit 332 corresponding to receiver stimulator unit 1022 of FIG. 1B) and an electrode assembly 348 (which may include an array of electrode contacts 148 of FIG. 1A). Power storage element 212 is configured to store power received by transceiver unit 208, and to distribute power, as needed, to the elements of implantable component 344A.

As shown, electronics module 322 includes a stimulator unit 332. Electronics module 322 can also include one or more other functional components used to generate or control delivery of electrical stimulation signals 315 to the recipient. As described above with respect to FIG. 1A, electrode assembly 348 is inserted into the recipient's cochlea and is configured to deliver electrical stimulation signals 315 generated by stimulator unit 332 to the cochlea.

In the embodiment depicted in FIG. 3A, the external device 304A includes a sound processor 310 configured to convert sound signals received from sound input unit 311 (e.g., a microphone, an electrical input for an FM hearing system, etc.) into data signals. In an exemplary embodiment, the sound processor 310 corresponds to data processor 210 of FIG. 2A.

FIG. 3B presents an alternate embodiment of a cochlear implant 300B. The elements of cochlear implant 300B correspond to the elements of cochlear implant 300A, except that external device 304B does not include sound processor 310. Instead, the implantable component 344B includes a sound processor 324, which may correspond to sound processor 310 of FIG. 3A.

As will be described in more detail below, while not shown in the figures, external device 304A/304B and/or implantable component 344A/344B include respective inductive communication components.

FIGS. 3A and 3B illustrate that external device 304A/304B can include a power source 213, which may be the same as power source 213 depicted in FIG. 2A. Power from power source 213 can be transmitted by transceiver unit 306 to transceiver unit 308 to provide power to the implantable component 344A/344B, as will be detailed below. FIGS. 3A and 3B further detail that the implantable component 344A/344B can include a power storage element 212 that stores power received by the implantable component 344 from power source 213. Power storage element 212 may be the same as power storage element 212 of FIG. 2A.

In contrast to the embodiments of FIGS. 3A and 3B, as depicted in FIG. 3C, an embodiment of a cochlear implant 300C includes an implantable component 344C that does not include a power storage element 212. In the embodiment of FIG. 3C, sufficient power is supplied by external device 304A/304B in real time to power implantable component 344C without storing power in a power storage element. In FIG. 3C, all of the elements are the same as FIG. 3A except for the absence of power storage element 212.

Some of the components of FIGS. 3A-3C will now be described in greater detail.

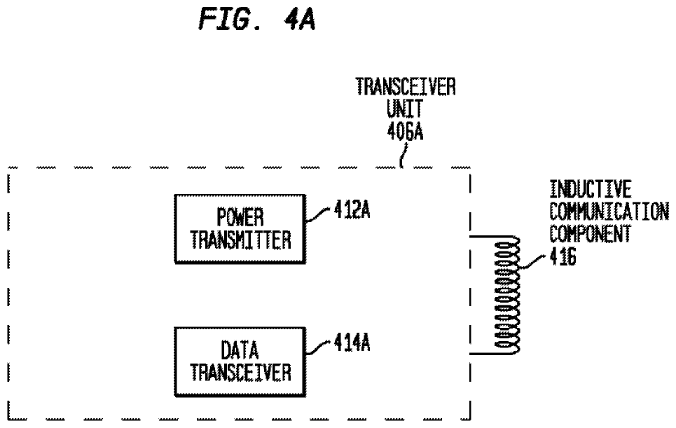

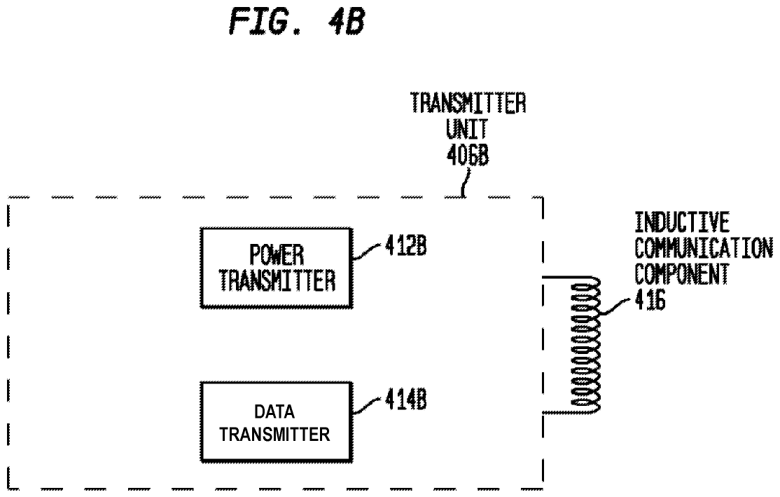

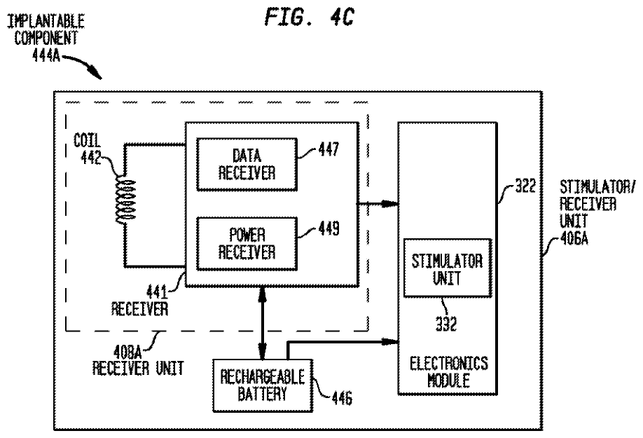

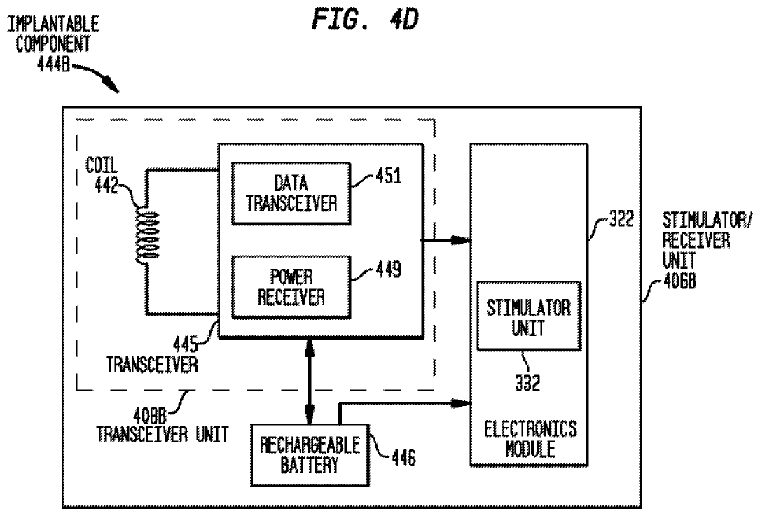

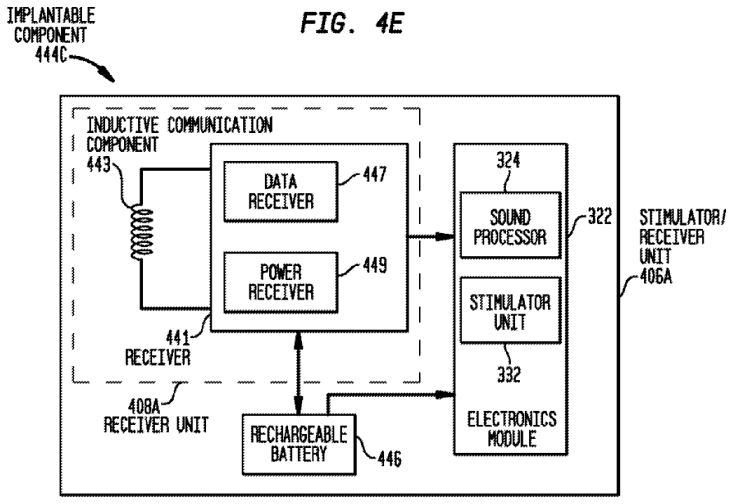

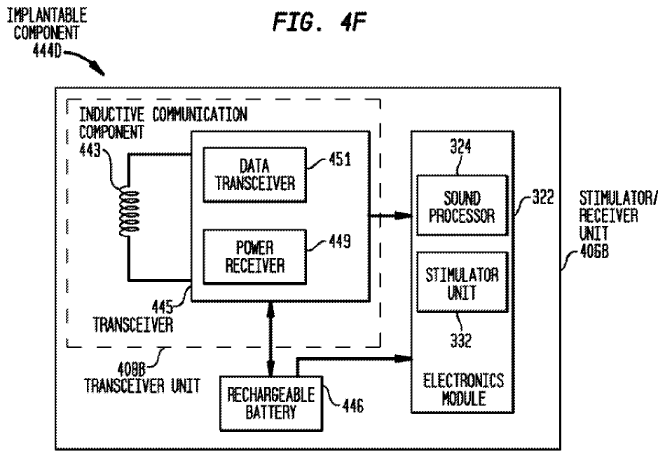

FIG. 4A is a simplified schematic diagram of a transceiver unit 406A in accordance with an embodiment. An exemplary transceiver unit 406A may correspond to transceiver unit 206 of FIGS. 2A-3C. As shown, transceiver unit 406A includes a power transmitter 412 a, a data transceiver 414A and an inductive communication component 416.

In an exemplary embodiment, as will be described in more detail below, inductive communication component 416 comprises one or more wire antenna coils (depending on the embodiment) comprised of multiple turns of electrically insulated single-strand or multi-strand platinum or gold wire (thus corresponding to coil 137 of FIG. 1B). Power transmitter 412A comprises circuit components that inductively transmit power from a power source, such as power source 213, via an inductive communication component 416 to implantable component 344A/B/C (FIGS. 3A-3C). Data transceiver 414A comprises circuit components that cooperate to output data for transmission to implantable component 344A/B/C (FIGS. 3A-3C). Transceiver unit 406A can receive inductively transmitted data from one or more other components of cochlear implant 300A/B/C, such as telemetry or the like from implantable component 344A (FIG. 3A).

Transceiver unit 406A can be included in a device that includes any number of components which transmit data to implantable component 334A/B/C. For example, the transceiver unit 406A may be included in a behind-the-ear (BTE) device having one or more of a microphone or sound processor therein, an in-the-ear device, etc.

FIG. 4B depicts a transmitter unit 406B, which is identical to transceiver unit 406A, except that it includes a power transmitter 412B and a data transmitter 414B.

It is noted that for ease of description, power transmitter 412A and data transceiver 414A/data transmitter 414B are shown separate. However, it should be appreciated that in certain embodiments, at least some of the components of the two devices may be combined into a single device.

FIG. 4C is a simplified schematic diagram of one embodiment of an implantable component 444A that corresponds to implantable component 344A of FIG. 3A, except that transceiver unit 208 is a receiver unit. In this regard, implantable component 444A comprises a receiver unit 408A, a power storage element, shown as rechargeable battery 446, and electronics module 322, corresponding to electronics module 322 of FIG. 3A. Receiver unit 408A includes an inductance coil 442 connected to receiver 441. Receiver 441 comprises circuit components which receive, via an inductive communication component corresponding to an inductance coil 442, inductively transmitted data and power from other components of cochlear implant 300A/B/C, such as from external device 304A/B. The components for receiving data and power are shown in FIG. 4C as data receiver 447 and power receiver 449. For ease of description, data receiver 447 and power receiver 449 are shown separate. However, it should be appreciated that in certain embodiments, at least some of the components of these receivers may be combined into one component.

In the illustrative embodiments, a receiver unit 408A and transceiver unit 406A (or transmitter unit 406B) establish a transcutaneous communication link over which data and power is transferred from transceiver unit 406A (or transmitter unit 406B), to implantable component 444A. As shown, the transcutaneous communication link comprises a magnetic induction link formed by an inductance communication component system that includes inductive communication component 416 and coil 442.

The transcutaneous communication link established by receiver unit 408A and transceiver unit 406A (or whatever other viable component can so establish such a link), in an exemplary embodiment, may use time interleaving of power and data on a single radio frequency (RF) channel or band to transmit the power and data to implantable component 444A. A method of time interleaving power according to an exemplary embodiment uses successive time frames, each having a time length and each divided into two or more time slots. Within each frame, one or more time slots are allocated to power, while one or more time slots are allocated to data. In an exemplary embodiment, the data modulates the RF carrier or signal containing power. In an exemplary embodiment, transceiver unit 406A and transmitter unit 406B are configured to transmit data and power, respectively, to an implantable component, such as implantable component 344A, within their allocated time slots within each frame.

The power received by receiver unit 408A can be provided to rechargeable battery 446 for storage. The power received by receiver unit 408A can also be provided for distribution, as desired, to elements of implantable component 444A. As shown, electronics module 322 includes stimulator unit 332, which in an exemplary embodiment corresponds to stimulator unit 322 of FIGS. 3A-3C, and can also include one or more other functional components used to generate or control delivery of electrical stimulation signals to the recipient.

In an embodiment, implantable component 444A comprises a receiver unit 408A, rechargeable battery 446 and electronics module 322 integrated in a single implantable housing, referred to as stimulator/receiver unit 406A. It would be appreciated that in alternative embodiments, implantable component 344 may comprise a combination of several separate units communicating via wire or wireless connections.

FIG. 4D is a simplified schematic diagram of an alternate embodiment of an implantable component 444B. Implantable component 444B is identical to implantable component 444A of FIG. 4C, except that instead of receiver unit 408A, it includes transceiver unit 408B. Transceiver unit 408B includes transceiver 445 (as opposed to receiver 441 in FIG. 4C). Transceiver unit 445 includes data transceiver 451 (as opposed to data receiver 447 in FIG. 4C).

FIGS. 4E and 4F depict alternate embodiments of the implantable components 444A and 444B depicted in FIGS. 4C and 4D, respectively. In FIGS. 4E and 4F, instead of coil 442, implantable components 444C and 444D (FIGS. 4E and 4F, respectively) include inductive communication component 443. Inductive communication component 443 is configured to vary the effective coil area of the component, and may be used in cochlear implants where the exterior device 304A/B does not include a communication component configured to vary the effective coil area (i.e., the exterior device utilizes a standard inductance coil). In other respects, the implantable components 444C and 444D are substantially the same as implantable components 444A and 444B. Note that in the embodiments depicted in FIGS. 4E and 4F, the implantable components 444C and 444D are depicted as including a sound processor 342. In other embodiments, the implantable components 444C and 444D may not include a sound processor 342.

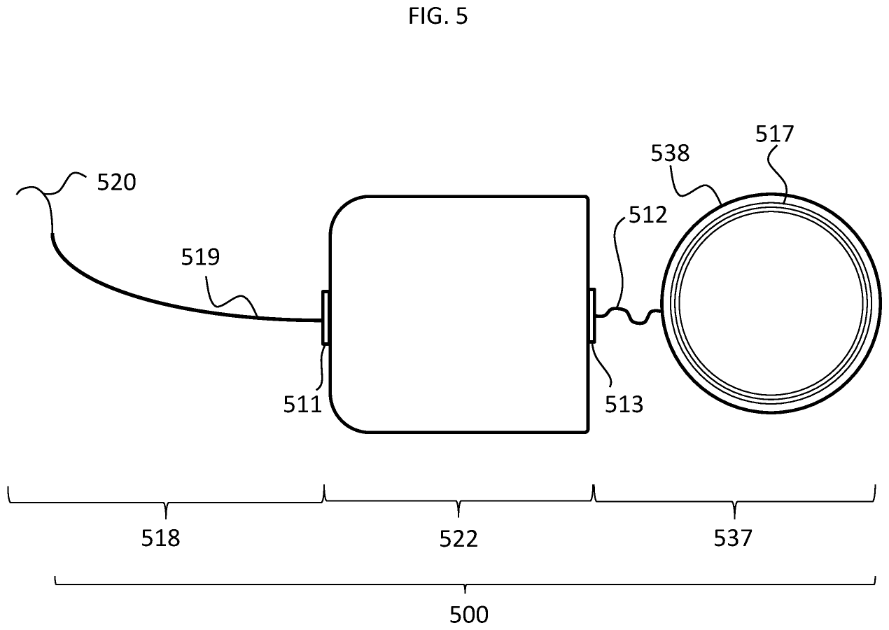

FIG. 5 depicts an exemplary alternate embodiment of an implantable component of a cochlear implant in a modularized form. Here, implantable component 500 corresponds to the implantable component 100 detailed above with respect to functionality and componentry, except that the electrode assembly is readily removable from the stimulator unit and the implantable coil is also readily removable from the stimulator unit (as opposed to the stimulator unit and the implantable coil being held together by the housing made of elastomeric material 199 as detailed above, and the elongate electrode assembly 118 being effectively permanently attached to the stimulator unit). More particularly, the implantable component 500 includes a receiver stimulator unit 522 that includes one or more feedthrough assemblies that permit signal communication with the coil 517 and the interior of the housing containing functional electronics of the cochlear implant, while maintaining hermetic sealing of that housing, and further includes one or more feedthroughs than enable communication with an electrode array to the receiver stimulator unit 522. In this regard, as can be seen, the implantable component 500 includes a coil unit 537 that includes a coil 517 located in a silicone body 538, and an electrical lead assembly 515 that is connected to a feedthrough 513 of the receiver stimulator unit 522, thus placing the coil 517 into signal communication with the electronic assembly of the receiver stimulator unit 522. On the opposite size of the stimulator unit 522 is feedthrough 511 of the receiver stimulator unit 522. Attached to the feedthrough 511 is the electrode assembly 518, which includes lead 519 to which is attached to electrode array 520 at the distal end thereof, the feedthrough 5111 placing the electrode array (via the lead 519) into signal communication with the interior of the receiver stimulator unit 522. In an exemplary embodiment, the connectors 510 and 512 are removable from the feedthroughs 511 and 513, respectively, thus enabling the electrode assembly 518 and the coil unit 537 to be removed from signal communication with the stimulator unit 522.

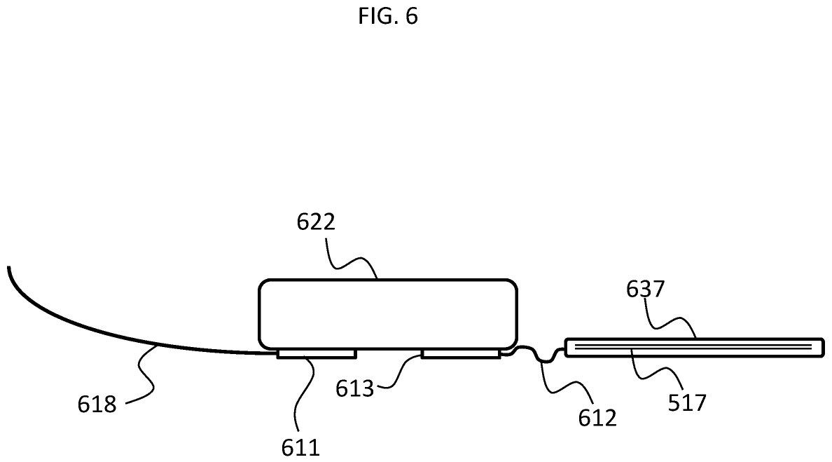

FIG. 6 depicts another exemplary alternate embodiment of an implantable component of a cochlear implant in a modularized form. As with implantable component 500, implantable component 600 corresponds to the implantable component 100 detailed above with respect to functionality and componentry. More particularly, the implantable component 600 includes a stimulator unit 622 that includes one or more feedthrough assemblies that permit removable attachment of the coil and the electrode array to the receiver stimulator unit 522. In this regard, as can be seen, the implantable component 600 includes a coil unit 637 that includes a coil 517 located in a silicone body, and an electrical lead assembly 612 that is connected to a feedthrough 613 of the receiver stimulator unit 622, thus placing the coil 617 into signal communication with the electronic assembly of the receiver stimulator unit 622. As can be seen, instead of the feedthrough 613 being on the side of the stimulator unit 622, it is on the bottom (the skull-facing side). Also, a feedthrough 611 of the receiver stimulator unit 622 is located adjacent feedthrough 613 on the bottom of the unit 622. Attached to the feedthrough 611 is the electrode assembly 618, which includes a lead to which is attached to electrode array at the distal end thereof, and includes connector 610 that is attached to feedthrough 611, thus placing the electrode array into signal communication with the stimulator unit 622.

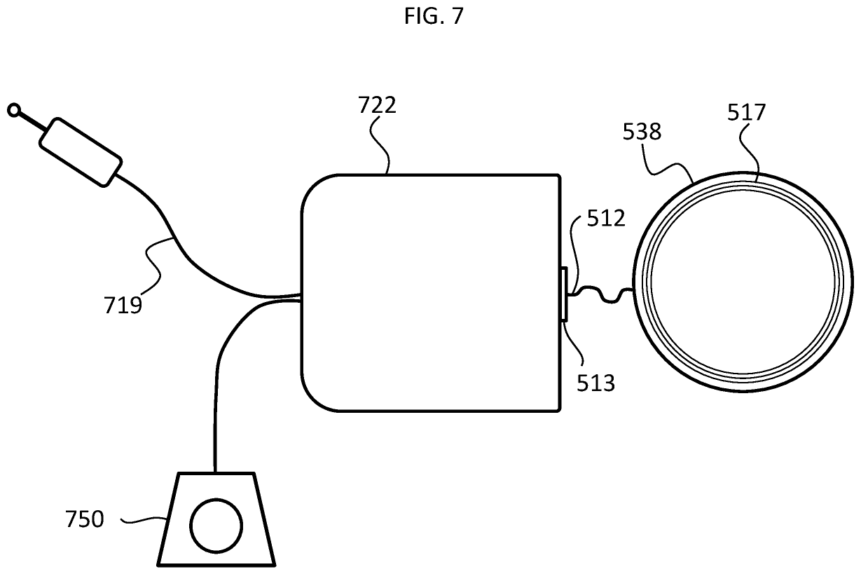

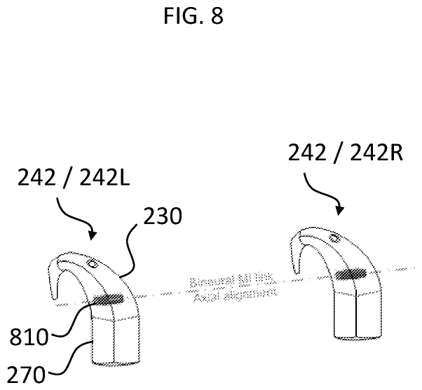

FIG. 7 depicts a totally implantable hearing prosthesis that includes a stimulating assembly 719 in the form of a DACS actuator (again, in keeping with the above, any disclosure of one type of output stimulating device corresponds to another disclosure of any other type of stimulation device herein, providing that the art enables such—thus, the disclosure of this DACS actuator corresponds to an alternate disclosure of a middle ear actuator or an active transcutaneous bone conduction device actuator, or a cochlear implant electrode array, or a retinal implant electrode array, etc., with the circuitry of the implant being different accordingly), and the hearing prosthesis further includes an implantable microphone 750. In this embodiment, the stimulating assembly and the implantable microphone are in signal communication with the electronics assembly located in receiver stimulator unit 722 via the same feedthrough or via separate respective feedthroughs. It is noted that the embodiment of FIG. 7 depicts a configuration where the feedthrough(s) are located on the bottom of the housing, and a feedthrough is also located on a side of the housing. It is noted that in some embodiments, all of the feedthroughs are located on the bottom of the housing. The embodiment of FIG. 7 is presented to show that the various configurations of feedthrough locations can be combined in some embodiments.

In view of the above, it is to be understood that in an exemplary embodiment, there is a device is hermetically sealed and is implantable, which includes a housing. The housing contains circuitry of a hearing prosthesis, and corresponds to the housing detailed above or variations thereof having opening(s) in which feedthrough assembly(ies) are located in the opening(s). The housing can also contain a battery so that the device can be “self powered” and thus be a totally implantable hearing prosthesis.

Embodiments include a modified version of the implantable component 100 as detailed above, and will be described below, but first, some background information on external components.

FIG. 2C presents additional details of an external component assembly 242, corresponding to external component 142 above.

External assembly 242 typically comprises a sound transducer 291 for detecting sound, and for generating an electrical audio signal, typically an analog audio signal. In this illustrative arrangement, sound transducer 291 is a microphone. In alternative arrangements, sound transducer 291 can be any device now or later developed that can detect sound and generate electrical signals representative of such sound. An exemplary alternate location of sound transducer 291 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 216 (sometimes referred to as a headpiece) comprises an external coil 228 (which can correspond to coil 130 of the external component of FIG. 1A) and, a magnet (not shown) secured directly or indirectly to the external coil 228. The signal processing unit processes the output of microphone 291 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals—because element 230 contains this circuitry, the entire component 230 is often called a sound processing unit or a signal processing unit. These coded signals can be referred to herein as a stimulation data signals, which are provided to external transmitter unit 296 via a cable 247. In this exemplary arrangement of FIG. 2C, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 2C that utilizes the bayonet connector.) Conversely, in some embodiments, there is no cable 247. Instead, there is a wireless transmitter and/or transceiver in the housing of component 230 and/or attached to the housing (e.g., a transmitter/transceiver can be attached to the receptacle 219) and the headpiece (transmitter unit 296) can include a receiver and/or transceiver, and can be in signal communication with the transmitter/transceiver of/associated with element 230.

FIG. 1E provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1E, an ITE component 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). ITE component 250 includes a housing 256, which can be a molding shaped to the recipient. Inside ITE component 250 there is provided a sound transducer 291 that can be located on element 250 so that the natural wonders of the human ear can be utilized to funnel sound in a more natural manner to the sound transducer of the external component. In an exemplary arrangement, sound transducer 242 is in signal communication with the remainder of the BTE unit via cable 252, as is schematically depicted in FIG. 1E via the sub cable extending from sound transducer 242 to cable 252. Shown in dashed lines are leads 21324 that extend from transducer 291 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal.

Also, FIG. 2C shows a removable power component 270 (sometimes battery back, or battery for short) directly attached to the base of the body/spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

The above description presents baseline technologies that are not innovative and do not form the basis of the invention herein. In at least some exemplary embodiments, the teachings above are used in combination with the innovative teachings below. Further, in at least some exemplary embodiments, the teachings above are modified so as to implement the innovative teachings below. In this regard, in at least some exemplary embodiments, the above is modified so as to enable the use thereof with the teachings herein.

FIG. 8 presents some additional features of the exemplary external assembly 242, along with an exemplary arrangement of use in a bilateral hearing prosthesis. Here, as can be seen, there is a left external assembly 242L and a right external assembly 242R. In this exemplary embodiment, the external assemblies correspond to those of FIG. 2C (some portions of the assemblies are not shown, such as the headpiece and the ITE component—it is noted that in some embodiments these components are optional and may not be present, and thus the arrangement of FIG. 8 can depict the outer profile of these devices somewhat accurately).

In this exemplary embodiment, as can be seen, the external assemblies 242 include cylindrical antennas (sometimes called rod antennas) 810. These are generally arrayed within the spine of the BTE device such that when utilized in the bilateral arrangement (conceptually shown in FIG. 8—there would be a human head in between the two devices and the BTE devices would extend from a front of the respective pinnas to behind the respective pinnas in a traditional manner), the axis about which the respective coils of the antennas are wound would lie on the same axis as shown/would be at least generally aligned. Roughly, the axis of windings of the antennas would be normal or roughly normal to the outside surfaces of the spine that face the skin over the mastoid bone when worn behind the ear. In an exemplary embodiment, the physical implementation of the external MI-radio antennas inside the external device in a bilateral system is typically well-defined. In an exemplary embodiment, the MI radio antennas are utilized to communicate between the two external components in a bilateral arrangement. Embodiments include MI radio antennas that are utilized to both communicate between the external components and the implanted components. In an exemplary embodiment, the antennas and the systems associated there with can be one way (send or receive) or can be two-way (send and receive). It is briefly noted that the concept of FIG. 8 would also be applicable, in at least some exemplary embodiments, to utilization of MI-radio in a bilateral system that utilizes in the ear devices, such as a totally in the ear device or an in-the-ear device where the MI radio antennas are located in the ear canal approximate thereto or otherwise on the side of the pinna opposite that which results when the behind the ear device arrangement is utilized of FIG. 8.

In this exemplary embodiment, the external assembly would include at least two antennas for transcutaneous communication—a first antenna of the headpiece/transmitter unit 216 for power transfer to the implantable component and a second antenna 810 for data transfer to the implantable component. In an exemplary embodiment, the devices are configured such that power is only transferred via the coil 228 and no data is transferred via that coil, and data is transferred via antenna 810 but no power is transferred via that antenna. In an exemplary embodiment, the external component can be configured to transmit data and power from the coil 228 so as to provide redundancy in the event that data cannot be transferred via antenna 810. Corollary to this is that in an exemplary embodiment, the implanted device can be configured such that the implant would not recognize data that is transmitted to the coil 137 that communicates with the external coil 228 and only transduces power that is received by the implanted coil 137. That said, in an exemplary embodiment, the implanted device can be configured such that the implant would recognize data that is transmitted to coil 137 in addition to transducing power, again to provide redundancy.

In an exemplary embodiment, such as where the implant is a partially implantable hearing prosthesis that relies on the external assembly to provide power in real time or near real time to operate (e.g., there is no implanted battery), power would be transmitted over the link established between the coil 228 and the coil 137, and the data to operate the implant would be transmitted over the link established on one side by antenna 810 and a corresponding implanted antenna that will be described in a moment. In this regard, the external assembly can correspond to that of FIG. 2C.

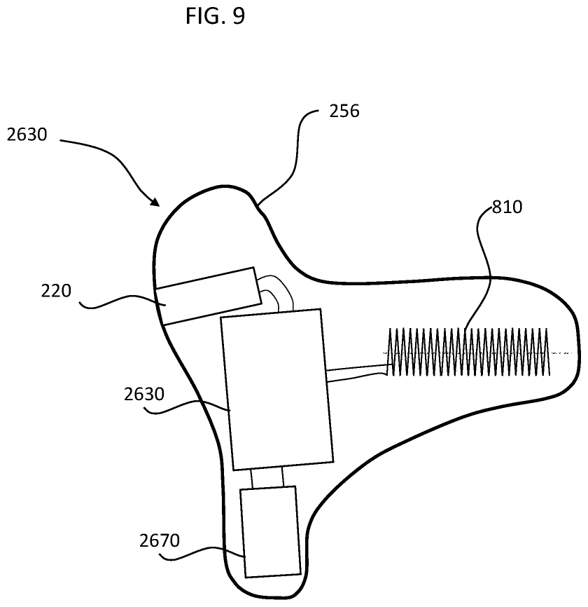

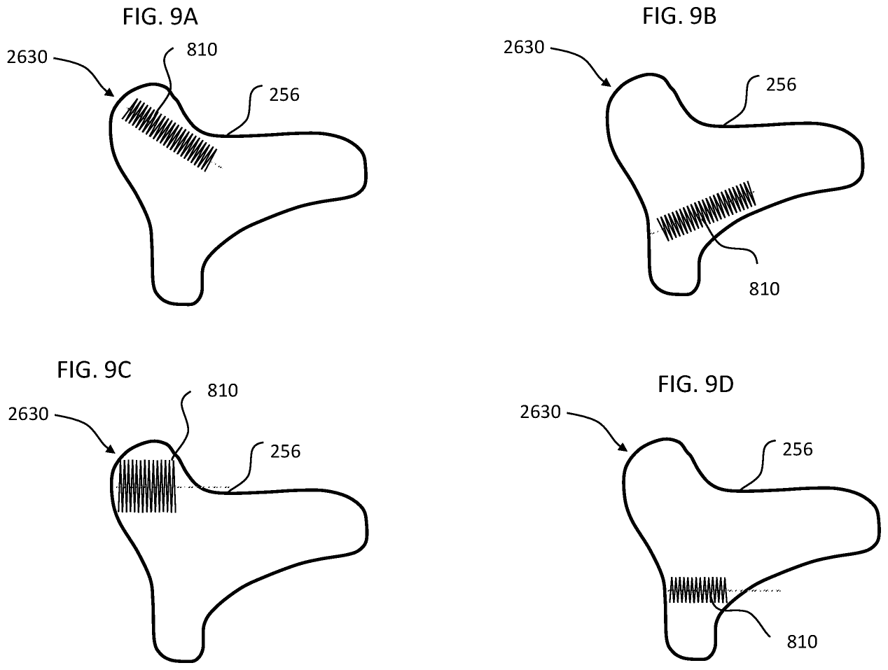

FIG. 9 presents another exemplary embodiment of an in-the-ear device 2630 having utilitarian value with respect to the teachings herein. This device is a fully contained external component of a cochlear implant or a middle ear implant or a DACS or an active transcutaneous bone conduction device. In this exemplary embodiment, a microphone 291 is supported by housing 256 which is in signal communication via leads to a sound processor 2630. In an exemplary embodiment, the sound processor 2670 can be a miniaturized version of the sound processor utilized with the embodiments detailed above, and can be a commercially available sound processor that is configured for utilization within an ITE device. As seen, there is a battery 2670 that provides power to the system. Consistent with the teachings above, there is a cylindrical antenna 810 that is in signal communication via leads with the sound processor 2630. In this exemplary embodiment, the ITE device 2630 communicates with the implanted component via MI radio in a manner concomitant with the teachings detailed herein with respect to the ITE device that is in signal communication with a BTE device.

It is briefly noted that the antenna 810 can be located in other portions of the behind the ear device. FIGS. 9A-9D depict some alternate placements of the antenna, and also showing alternate configurations of the antenna where a wider antenna that is shorter can be utilized depending on the placement.

It is briefly noted that while the embodiments of FIGS. 9A-D show a complete external sound processor device that is completely embodied in an ITE device, in an alternate exemplary embodiment, the ITE device can be in wired communication to the BTE device. In this regard, this can enable at least some of the components of the embodiments of FIGS. 9 to 9D, such as the battery and/or the sound processor, to be located in the spine of the BTE device. In an exemplary embodiment, the ITE device is utilized to place the coil antenna 810 at the desired location (it can also be utilized to place the microphone at a desirable location consistent with the teachings above). Accordingly, the embodiments of FIGS. 9A-D can be utilized in conjunction with a BTE device. Also, wired communication may not necessarily be needed. In an exemplary embodiment, a wireless transmitter/receiver arrangement can be utilized to wirelessly transmit data from the BTE device to the ITE device. In this exemplary embodiment, the ITE device can include an onboard battery.

It is also noted that in some exemplary embodiments include an MI radio antenna located in an OTE (off the ear) device. In an exemplary embodiment of this arrangement, this is a device that is located and otherwise magnetically held over the implanted wide diameter coil 137 of the implant, and does not have a component that is in contact with the pinna that is physically connected to the OTE device. There could be such a device that is in radio signal communication there with, and there could be an ITE device that is in radio signal communication therewith, but there is no physical link between the two—the link is electromagnetic. To be clear, any disclosure herein with respect to functionality and/or structure of a BTE device corresponds to an alternate disclosure of such with respect to an ITE device and an OTE device and vice versa two more times, unless otherwise noted and unless the art does not enable such.

It is also noted that the various antenna orientations and geometries shown in FIGS. 9A-D can also be applicable to the antennas of the BTE device. As will be detailed below, the teachings detailed herein permit the establishment of a viable and otherwise high quality and/or well-defined magnetic radio inductance link between a given implant in a wide variety of designs of external components located in a wide variety of orientations.

While the embodiment depicted in FIG. 9 is configured to operate autonomously from any other external component, in an alternative embodiment, one or more of the components of the embodiment of FIG. 9 can be utilized with the ITE device of the external assembly 242. In this regard, in an exemplary embodiment, instead of or in addition to the cylindrical antenna being located in the spine of the BTE device, the cylindrical antenna can be located in the ITE component (e.g., along with a microphone supported by the housing 2630).

Antenna 810 is part of a magnetic inductance radio (MI radio) system that enables the establishment of a utilitarian ipsilateral communication link between the external component and the implant device. The communication link may operate between 148.5 kHz and 30 MHz by way of example only and not by way of limitation (the link between the coil 137 and coil 130 can be, in some embodiments by way of example only and not by way of limitation, less than 30 MHz, such as between 3 and 15 MHz in general, and more specifically, 4.5 MHz and 7 MHz. The physical implementation of the external MI-radio antennas inside the external sound processor devices and hearing aids in a bilateral/binaural system can be as presented above, where the axis of symmetry of these cylindrical antenna rods are aligned between the two external devices (meaning their ‘axes of symmetry’ are ideally on a same line, as seen in FIG. 8 (and the alignment would also be the case with the embodiment of FIG. 9 when used in the bilateral scenario)).

It is noted that the teachings herein, while generally described in terms of transcutaneous communication, are also applicable to subcutaneous communication. That is, embodiments can be applicable to communication between two different antennas that are both implanted with in a recipient. This can be, for example, where there is utilitarian value with respect to maintaining a hermetic body, such as a housing, without the risk of utilizing a feedthrough or the like therethrough. By way of example only and not by way limitation, an antenna within a ceramic housing also containing a processor can communicate with a separate component that includes an implanted microphone. The utilization of the antenna in the housing can avoid the need for a feedthrough or the like from the component with the implanted microphone. Accordingly, any disclosure herein relating to transcutaneous communication also corresponds to a disclosure of subcutaneous communication unless otherwise noted providing that the art enable such.

With the above as background, embodiments of some teachings are such that the physical implementation of the MI-radio antennas of the implant for ipsilateral communication with the external component are well-defined as such to provide, and in some instances, guarantee, strong incoming MI implant signals. Embodiments can avoid communication interruptions caused by external interference and/or can avoid antenna positions/orientations in radio dead zones (dead spots) or close to radio dead zones. As used herein, the phrase dead zones means a zone where the in ingoing and outgoing magnetic flux in the enclosed area of an antenna (geometric median of all enclosed winding surfaces of a given antenna cancels each other out to zero or effectively zero or a zone where the magnetic field lines do not cross the enclosed area i.e., the field line vectors are parallel to the enclosed winding surface). Some additional details of this will be described below.

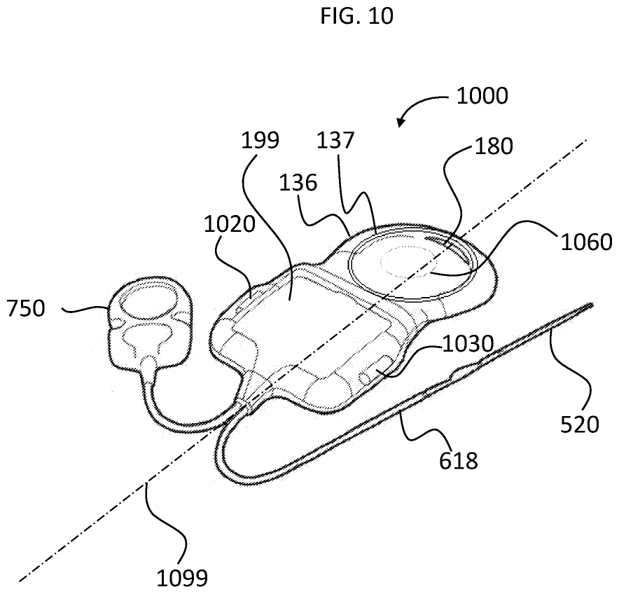

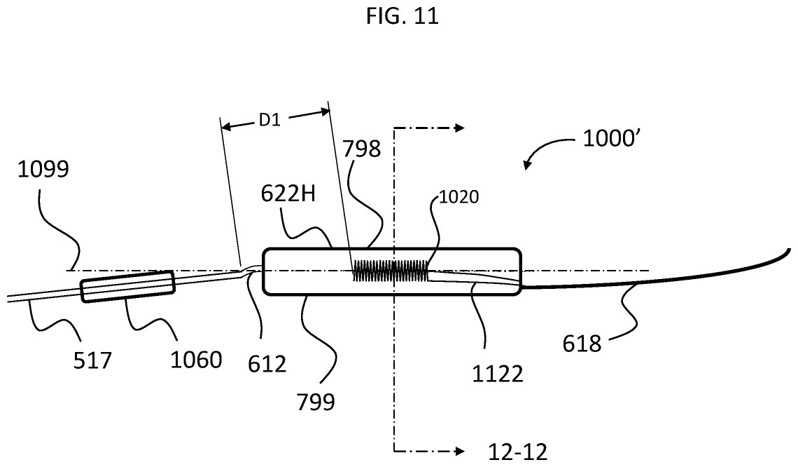

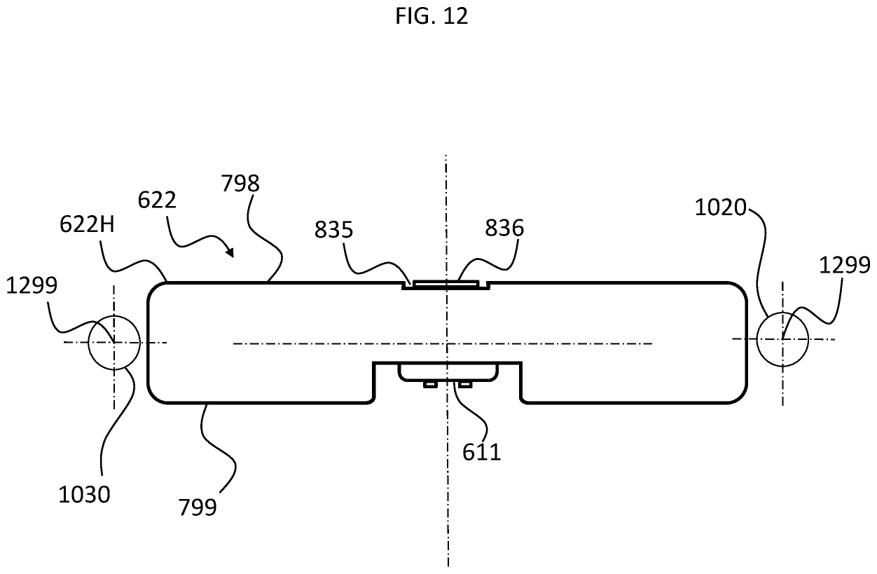

Accordingly, in an exemplary embodiment, as seen in FIGS. 10-12, there is an implantable component 1000, with some of the reference numbers reused to demonstrate like components. FIG. 10 depicts an isometric view of the component 1000, along with the longitudinal axis 1099 of the receiver-stimulator for future reference. For example, there is the silicone overmold 136 and 199, overmolding the coil number 137 and the housing of the receiver stimulator (the housing is not seen in FIG. 10, but seen in FIGS. 11 and 12—FIGS. 11 and 12 do not depict the silicone for purposes of clarity). Briefly, it is noted that magnet apparatus 1060 is an exemplary cassette magnet assembly that includes a magnet located in a housing or a set of plates that are encased in the silicone 136. This magnet apparatus 1060 can be removed by a slit 180 as noted above. More particularly to the embodiments herein, as can be seen, cylindrical antennas 1020 and 1030 are located on flanks of the implantable component 1000 proximate the housing of the receiver stimulator. FIG. 11 depicts a side view of the implantable component 1000 without the silicone overmold (hence the assembly has been identified as 1000′— what is shown can be considered an embryonic implantable component 1000, awaiting silicone overmolding). Here, the housing 622H of the receiver stimulator can be seen, along with the cylindrical coil antenna 1020 (where antenna 1030 is located on the opposite side of the housing 622H, and thus eclipsed by the housing 622H). Leads 1122 can be seen extending from antenna 1020, which leads extend to the feedthrough 611 (FIG. 12 does not depict the attachment of the leads to the feedthrough for clarity). The leads 1122 place the antennas into signal communication with the electronic components of the receiver stimulator.

FIG. 10 depicts how a silicone body or any other type of biocompatible material such as ceramic or peek material can support the antennas 1020 and 1030. In an exemplary embodiment, the antennas are encased in silicone/silicone is overmolded over the antennas, thus supporting the antennas during use and otherwise attachment to the remainder of the implantable component 1000. Additional details of this will be described below.

As noted, stimulator 622 includes a housing 622H which supports the feedthroughs 611 (and another feedthrough eclipsed thereby). Depicted are electrical contacts extending out the bottom of the feedthrough 611. As seen, bottoms of the feedthroughs are recessed relative to the bottom surface 799. FIG. 12 depicts a cross-sectional view of stimulator unit 622 and the antennas 1020 and 1030. As can be seen, a top surface 798 is parallel to the bottom surface 799, although in other embodiments, the top of the housing 622H may not be parallel to the bottom surface 799 (the top could be a dome-shaped component). Also seen is a return electrode/electrode plate 836. The housing 622H is depicted cut in half (but the innards are not depicted), with housing wall 940 (through which the view of FIG. 11 is cut).

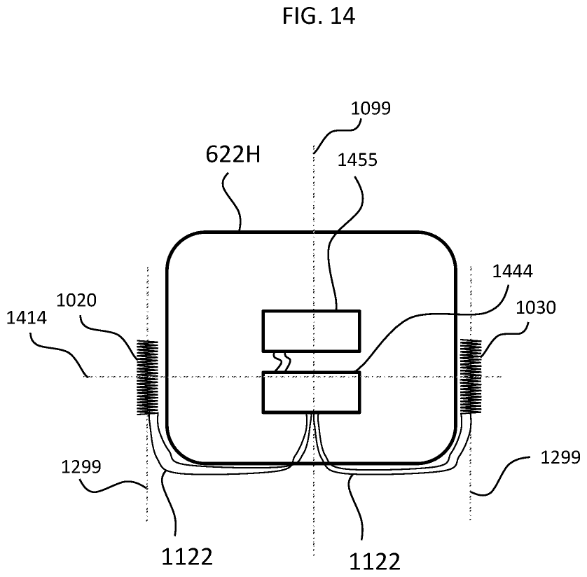

As will be further explained below, antennas 1020 and 1030 are placed so that they form a symmetrical implant MI radio antenna system (the coil 137 is not part of this system for the purposes of this discussion, but can be—the coil 137 need not have any of the symmetrical features disclosed herein in at least some embodiments, with respect to the symmetry features associated with the MI radio as detailed herein) for short range radio communication purposes residing outside of the housing of the receiver stimulator, which can be a titanium housing in some embodiments that acts as an electromagnetic shield and is not radio transparent. Here, the implant MI radio antenna system comprises two antenna coils that are in signal communication with an MI-radio transceiver system located inside the housing of the receiver stimulator. Here, in this embodiment, both coil antennas of the antenna system are placed in series and are in signal communication with one single MI-radio transceiver (e.g., transceiver 1444 as seen in FIG. 14—more on this below) inside the housing of the receiver stimulator via feedthroughs. That said, in an alternate embodiment, respective antennas of this MI radio antenna system are in signal communication with respective individual MI-radio transceivers. The two antenna coils are placed in a symmetrical way aside the implant body. Here, as shown, the two antenna coils are placed at the center of the left and the right longitudinal sidewalls outside of the housing. The MI radio implant antenna coils have in some embodiments round, rectangular or oval cross sections (winding cross-sections): short or long coils may be used (Short=low number of windings, higher diameter; Long=High number of windings, lower diameter). Any arrangement of antennas that can have utilitarian value with respect to the teachings detailed herein can be utilized in at least some exemplary embodiments. In at least some exemplary embodiments, the antennas are made of platinum, gold, doped gold, etc. In some embodiments, there is no ferrite core about which the windings are wound, while in other embodiments there is such. When a ferrite core is utilized, in some embodiments, the winding-core assembly or the core assembly alone can be encased in ceramic and/or peek material, where with respect to the former, the leads to the coil can extend out of the encasement. In an exemplary embodiment, the implant is configured to place one of the second antenna or the third antenna into signal communication with the transceiver to the exclusion of the other of the second antenna or third antenna. This can be done automatically based on an autonomous analysis of signals and/or spatial positioning of one or more components of the system. This can also be done based on input received from, for example, the external component or some other programming device or the like.

In view of the above, it can be seen that in an exemplary embodiment, there is a device, comprising an implantable hermetically sealed biocompatible housing, such as housing 622H of the receiver stimulator unit detailed above. The device further includes electronics located inside the housing. Additional details of the electronics will be provided below. The device further includes a first implantable antenna coil, the first implantable antenna coil electrically connected to the electronics and/or the device is configured to connect the first implantable antenna coil to the electronics (e.g., via a switch—note that if the switch is closed, the first coil is electrically connected to the electronics (assuming there is no other break), and a second implantable antenna coil, the second implantable antenna coil electrically connected to the electronics and/or the device is configured to connect the second implantable antenna coil to the electronics. In some embodiments of this device, the electronics includes magnetic induction radio communication circuitry, the device is configured to enable a magnetic induction communication link, and the device is implantable in a human recipient between skin and skull bone of the human.

In some embodiments of this device, the electronics includes at least two separate transceivers and the first implantable antenna coil and the second implantable antenna coil are respectively electrically connected to respective transceivers of the at least two separate transceivers. In some embodiments, again where the electronics includes at least one transceiver, the implantable component is configured so that the first implantable antenna coil and the second implantable antenna coil can be electrically connected to and electrically disconnected from the at least one transceiver. In some embodiments, the magnetic induction radio circuitry includes at least one transceiver and the implantable component includes switching circuitry configured to respectively place the first implantable antenna coil and the second implantable antenna coil into electrical connection and electrical disconnection with the at least one transceiver.

In an exemplary embodiment of the device, the first implantable antenna coil and the second implantable antenna coil reside at separate locations and have respective longitudinal axes that are parallel to each other or are quasi-aligned with each other. Additionally, in some embodiments, the device includes a third antenna coil behind or above a pinna of the recipient allowing transcutaneous power transfers to the implantable component.

Also, in view of the above, embodiments include a device comprising an implantable housing, such as housing 622H of the receiver stimulator unit detailed above. In an exemplary embodiment, there are electronics located in the housing. In this exemplary embodiment, the electronics can correspond to the receiver stimulator electronics of the receiver stimulator unit of a cochlear implant. In this exemplary embodiment, at least some of the electronics can be configured to output a signal to a location outside the housing. In this regard, in an exemplary embodiment, this can correspond to the electronics circuitry of the stimulator portion of a receiver stimulator of a cochlear implant. The electronics can be the electronics of the receiver stimulator unit.

In this exemplary embodiment, there can be a first antenna, corresponding to the implantable coil number 137 of the implantable component 1000, which first antennas located outside the housing (this would be the third antenna coil behind or above the pinna allowing transcutaneous power transfers, noted in the paragraph immediately before the paragraph just above). This first antenna can be an inductance coil, such as a transcutaneous inductance coil. In this exemplary embodiment, the first antenna is in signal communication with the electronics (thus in signal communication with the receiver electronics portions of the receiver stimulator unit). It is briefly noted that the phrase electronics as used herein includes an integrated circuit as well as separate circuits. For example, the receiver electronics can be a separate component from the stimulator electronics (collectively they can be arranged in a unit). Conversely, the receiver electronics can be combined with the stimulator electronics on one single printed circuit board for example. Any implementation that will provide functionality of a receiver and the functionality of a stimulator of a receiver stimulator unit of a cochlear implant or any other alternate hearing prosthesis can be utilized in at least some exemplary embodiments.

Still further, in an exemplary embodiment, there can be seen a second antenna and a third antenna located outside the housing. Both of these antennas can be in signal communication with the electronics located inside the housing. The antennas can correspond to antennas 1020 or 1030 or any the other antennas detailed herein. And of course, consistent with the utilitarian value of the teachings detailed herein, this device can be implantable in a human recipient (i.e., it is made of bio compatible components, at least with respect to those that interface with body tissue and fluids inside the body).

Consistent with the teachings detailed herein, the second and third antennas are part of a magnetic inductance radio system (which can have a unified transducer or receiver or can have separate transducers or receivers).

Consistent with the teachings above, the first antenna in at least some exemplary embodiments is a different type of antenna than the second and third antennas. By way of example only and not by way of limitation, the first antenna is a wide diameter inductance coil, and the second and third antenna are narrow diameter inductance coils.

In an exemplary embodiment, the first antenna (e.g., coil 137) has an outermost maximum coil diameter of at least or equal to 15, 16, 17, 18, 19, 20, 21, 22, 23, 24, 25, 26, 27, 28, 29, 30, 31, 32, 33, 34, 35, 36, 37, 38, 39, or 40 mm, or any value or range of values therebetween in 0.1 mm increments (e.g., 23.3 mm, 27.1 mm, 18.8 to 36.5 mm, etc.). In an exemplary embodiment, the second and/or third antennas (they can have the same configurations, but can also be different) can have an outermost maximum coil diameter equal to or no greater than 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.5, 5, 5.5, 6, 6.5, 7, 7.5, or 8 mm, or any value or range of values therebetween in 0.005 mm increments). With respect to the embodiments where the antennas are cylindrical/cylinder antennas (e.g., the windings of the coils extend in a manner akin to cylinder walls, the height (as in cylinder height) can be equal to or no greater than 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.5, 10, 10.5, 11, 11.5, 12, 12.5, 13, 14, or 15 mm, or any value or range of values therebetween in 0.05 mm increments.

In at least some exemplary embodiments, at least X percent, such as a majority, of the conductive componentry of the second and/or third antenna by mass thereof is located within 1.5, 1.6, 1.7, 1.8, 1.9, 2, 2.1, 2.2, 2.3, 2.4, 2.5, 2.6, 2.7, 2.8, 2.9, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.5, 10, 11, 12, 13, 14, 15, 16, 17, 18, 19, or 20 mm, or any value or range of values therebetween in 0.01 millimeter increments from an outer surface of the housing of the receiver stimulator. X can be 55, 60, 65, 70, 75, 80, 85, 90, 95, or 100. In an exemplary embodiment, the most distal portion of the second and/or third antenna from the housing falls within the aforementioned distance from the housing.

In at least some exemplary embodiments, at least X percent, such as a majority, of the conductive componentry of the first antenna by mass thereof is located further than or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mm or any value or range of values therebetween in 0.1 millimeter increments from at least X percent of the conductive componentry of the second and/or third antenna by mass thereof. In an exemplary embodiment, the closest portion of a coil of the second and/or third antenna to a coil of the first antenna is greater than the just detailed values. FIG. 11 depicts by way of example a clearance with distance D1 measuring the closest portions that can correspond to any of the just mentioned distances in at least some exemplary embodiments (further than or equal to). As seen, this does not take into account the leads 612, and would not take into account the leads 1122 if such extended towards the coil 517 as opposed to away from the coil 517.

As seen, the MI radio antennas have a height (in terms of a cylinder—one would refer to this as a length in other situations) that is less than the length of the housing on a side thereof facing the antennas. In some embodiments, the height of a second antenna and/or third antenna is no more than or equal to 10, 15, 20, 25, 30, 35, 40, 45, 50, 55, 60, 65, 70, 75, 85, 90, 95, 100, 110, 120, or 130 percent, or any value or range of values therebetween in 1 percent increments of the length of the sidewall of the housing of the receiver stimulator to which it is closest. Also, as seen, the second antenna and/or the third antenna has a mean or median outermost diameter is less than a height of the closest sidewall of the housing. In some embodiments, the aforementioned diameter of the second antenna and/or third antenna is no more than or equal to 10, 15, 20, 25, 30, 35, 40, 45, 50, 55, 60, 65, 70, 75, 80, 85, 90, 100, 110, 120, or 130 percent or any value or range of values therebetween in 1 percent increments of the height of the sidewall of the housing of the receiver stimulator to which it is closest.

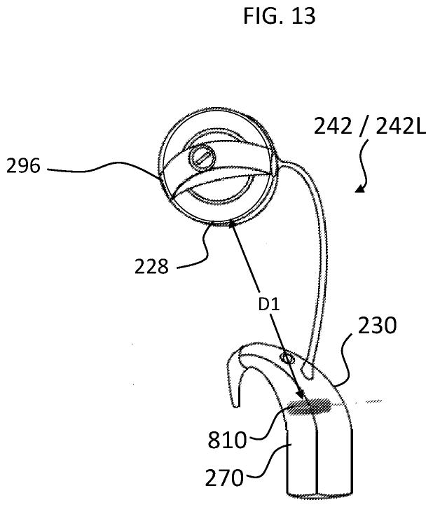

It is briefly noted that many of the aforementioned values are applicable to the antennas of the external component where applicable. For example, the aforementioned values associated with the distances between the large diameter coil and the MI coil of the implant could also be applicable to the corresponding coils of the external component (the headpiece antenna relative to the antenna 810). By way of example, FIG. 13 depicts the dimension D1 with respect to an external component in use attached to a 20 to 80 percentile or any percentile therebetween in 1% increments male and/or female born in the United States of America of 40 years of age as of Sep. 15, 2020. Conversely, the features associated with the housing of the receiver stimulator would not be present in the external component because there is no receiver stimulator component and the external component. But it is noted that the values for D1 for the external component can likely be different than those of the implanted component owing to the fact that the location of the MI antenna in the external component relative to the large diameter coil will be different than that with respect to the implantable component.

In an exemplary embodiment of the device just detailed and/or variations thereof, the second and third antennas are arranged symmetrically relative to the housing. This symmetry can be with respect to the left and right sides of the housing (where a plane parallel to the longitudinal axis 1099 bifurcates the left and right side and the second and third antennas are on the respective sides). FIG. 12 depicts this by way of example. This symmetry can also be respect to the height and/or the length of the housing, just as seen in FIG. 11, where the antenna 1030 on the opposite side mirrors that seen in FIG. 11.

In an exemplary embodiment, the second and third antennas are cylindrically coiled antennas that are arranged symmetrically relative to one another. Again, this is seen in FIGS. 11 and 12.

In an exemplary embodiment, the aforementioned second and third antennas are coiled antennas that have respective longitudinal axes that are parallel to one another. FIGS. 12 and 14 depict the longitudinal axes 1299 of the antennas 1020 and 1030 (FIG. 14 depicts a top view of the stimulator housing 622, also showing a transceiver 1444 for the second and third antennas that are connected via leads from the antennas extending to the feedthrough is in the bottom of the housing, along with a stimulator 1455 of a cochlear implant or other pertinent implanted hearing prostheses—shown are the coils 1020 and 1030 (second and third antennas) arranged such that they are on opposite sides of the housing 622H). In an exemplary embodiment, the longitudinal axes are within 15, 14, 13, 12, 11, 10, 9, 8, 7, 6, 5, 4, 3, 2, 1 degrees, or any value or range of values therebetween in 0.1° increments of each other as measured in any one of the frames of references of FIG. 11, 12 or 14. As seen, in FIGS. 11 (where antenna 1030 is located on the opposite far side and mirrors antenna 1020), 12 and 13, the longitudinal axes are parallel in all three frames of reference.

In some embodiments, the second and third antennas are arranged symmetrically relative to a first plane that lies on and is parallel to a longitudinal axis of an assembly comprising the housing and the first antenna (or just the housing or the first antenna in some embodiments). This is seen in FIG. 10 and FIG. 14, where first antenna (coil 517), not shown, would be symmetrical about axis 1099, and the plane would be normal to the page (of FIG. 14). In some embodiments, misalignment of the two antennas relative to the plane would be less than or equal to 15, 14, 13, 12, 11, 10, 9, 8, 7, 6, 5, 4, 3, 2, or 1%, or any value or range of values therebetween in 0.1% increments on a component portion to component portion basis (e.g., rearmost left outboardmost portion of antenna 1020 vs. rearmost right outboardmost portion of antenna 1030—a distance from the plane for one would be a value that no more or no less than 10% of a difference of the other). As seen, in this embodiment, the second and third antennas are on opposite sides of the housing (as will be detailed below in an alternate embodiment, the second and third antennas can be on the same side of the housing—more on this in a moment).

In an exemplary embodiment, again where the antennas are on opposite sides of the housing, the second and third antennas are arranged symmetrically relative to a first plane that lies on and is parallel to a longitudinal axis of an assembly comprising the housing and the first antenna (or just the housing or the first antenna in some embodiments), and the second and third antennas and the housing are arranged symmetrically relative to a second plane (1414—the plane extends out of the page—1414 can also represent a lateral axis of the housing 622H) normal to the first plane and normal to the longitudinal axis. This is seen in FIG. 14, where the first antenna (coil 517, not shown) would be symmetrical about axis 1099, and the plane would be normal to the page. In some embodiments, misalignment of the two antennas relative to the second plane would be less than or equal to 15, 14, 13, 12, 11, 10, 9, 8, 7, 6, 5, 4, 3, 2, or 1%, or any value or range of values therebetween in 0.1% increments on a component portion to component portion basis (e.g., the height (in cylindrical terms) of antenna 1020 vs. the height (again, in cylindrical terms) of antenna 1030 above plane 1414—is no more or no less than 10% of a difference of the other).

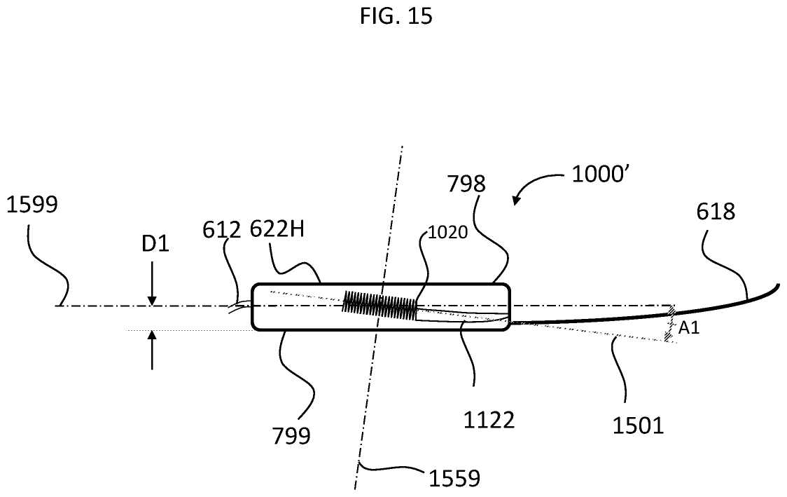

In an exemplary embodiment, the second and/or third antennas (1020 and/or 1030) are at an oblique angle relative to a plane of extension of the width and the length of the housing 622H. thus, an embodiment includes antenna coils that are tilted relative to the housing. This can be seen in FIG. 15, which depicts the height and the length of the housing (the height is the vertical direction, and the length is the horizontal direction). In an exemplary embodiment, that plane can be normal to the direction of extension of the height of the housing. In this regard, the longitudinal axis 1599 of the housing can represent the aforementioned plane, where the plane extends parallel to that line in and out of the page normal to the page. As seen, the longitudinal axis 1501 of the antenna 1020/the axis of winding 1501 (which can be the geometric center of the antenna and/or the center of mass of the antenna—more on this in a moment)—1501 can correspond to axis 1299 detailed above—of that antenna extends at an angle A1 relative to the longitudinal axis of the housing 1599 (or relative to a bottom and/or a top plane of the housing 622H and/or a plane upon which the maximum diameter of the housing extends in some other embodiments). A1 can be plus or minus (the orientation shown in FIG. 15 is plus) and equal to or less than or greater than 1, 2, 3, 4, 5, 6, 7, 7, 8, 10, 11, 12, 13, 14, 15, 20, 25, 30, 35, 40, or 45 degrees or any value or range of values therebetween in 0.1° increments. Also, the angle can be a generic angle which means that there is no plus or minus, it just is an angle that is oblique relative to the plane (it could be as shown in FIG. 15 or the mirror image of that about line 1599). Of course, in the embodiments where the angle is not oblique, the angle A1 can be zero.

It is noted that in some embodiments, the angle A1 (and as will be seen below, angle A2) of the second antenna can be different than the angle of the third antenna. Indeed, in an exemplary embodiment, one angle can be positive in one angle can be negative. That said, in an exemplary embodiment, the symmetrical arrangements on at least two planes can have utilitarian value.

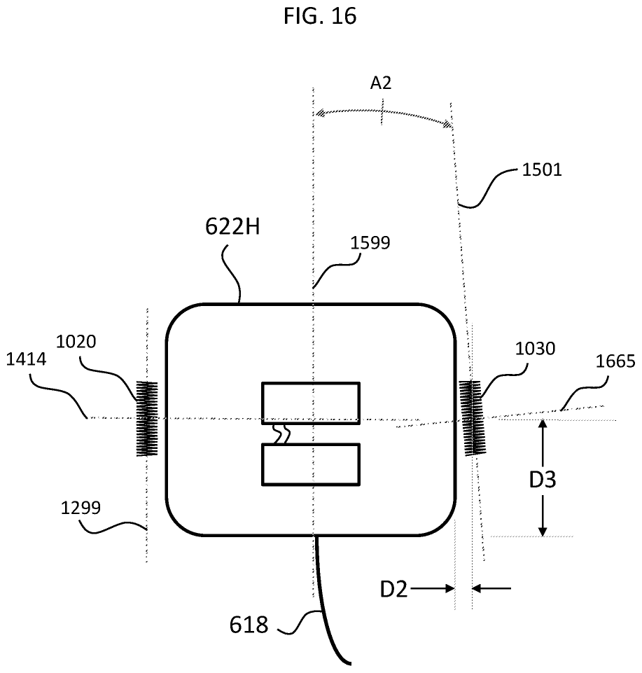

In an exemplary embodiment, the second and/or third antennas are at an oblique angle relative to a plane of extension of a height and a length of the housing. This can be seen in FIG. 16, which shows the length and the width of the housing 622H. The aforementioned plane can be planes that lie on and are parallel to the longitudinal axis 1599 extending out of the page and normal to the page. In an exemplary embodiment, the plane can be normal to the width. Note that the length as used herein corresponds to the measurement of the housing as seen in FIG. 1C in the horizontal direction. In an exemplary embodiment, angle A2 can be any of the angles A1 just detailed (they need not be the same, A1 and A2 can be different). Angle A2 as positive is shown in FIG. 16 (note that the antenna 137/the large diameter antenna would be located above the housing in the view shown in the FIG. 16). Angle A2 for the second antenna can be different than that for the third antenna. The angles can also be the same in the symmetric embodiments on two planes.

Thus, in view of the above, it can be seen that in an exemplary embodiment, the second and third antennas are arranged symmetrically relative to a first plane that lies on and is parallel to a longitudinal axis of an assembly comprising the housing and the first antenna, and the second and third antennas are located on a same side of the housing. Also in view of the above, it can be seen that in some embodiments, the second and third antennas are arranged symmetrically relative to a first plane that lies on and is parallel to a longitudinal axis of an assembly comprising the housing and the first antenna or just the housing or just the first antenna (any reference herein to symmetry associated with one of these three arrangements corresponds to a disclosure of symmetry with respect to the other two arrangements). Here, the second and third antennas are located on opposite sides of the housing, and the second and third antennas and the housing are arranged symmetrically relative to a second plane normal to the first plane and normal to the longitudinal axis.

FIGS. 15 and 16 depicts some additional data points associated with some embodiments. More specifically, as can be seen, there is an axis 1559 which corresponds to an axis that extends through a center of mass and/or a geometric center of the antenna 1020 and/or a location where a number of coils on one side equals a number of coils on the other and/or fractions thereof (this does not include the leads that extend to the antenna—here, the antenna begins where the coiling begins). This axis is normal to the axis 1501, which also extends through the center of mass and/or a geometric center of the antenna 1020 and/or the axis of winding of the antenna. (Note herein that reference to antenna 1020 corresponds to a disclosure associated with the other antenna, antenna 1030, and vice versa.) The crosspoint of the axis 1559 and 1501 can be located in the vertical direction D1 from the bottom surface 799 of the housing 622H (the surface that faces the skull when implanted). D1 can be less than or equal to or greater than 2, 3, 4, 5, 6, 7, 8, 9, or 10 mm, or any value or range of values therebetween in 0.1 mm increments. It is also noted that while the dimension D1 has been shown as being measured from the bottom of the housing, in an alternate embodiment, the dimension D1 can be measured from the top of the housing. Is also noted that in an exemplary embodiment, the dimension D1 can be measured from the topmost portion or the bottom most portion of the housing with respect to the frame of reference of FIG. 15.

It is also noted that with respect to FIG. 15, the highest most portion of the antenna can be less than or greater than or equal to 4, 5, 6, 7, 8, 9, 10, 11, 12, 13, 14, 15, 16, 17, 18, 19, or 20 millimeters, or any value or range of values therebetween in 0.1 mm increments from the bottom and/or the lowest most portion of the antenna can be less than or greater than or equal to 0, 1, 2, 3, 4, or 5 mm, or any value or range of values therebetween in 0.1 mm increments from the bottom.

Also, as seen, there is an axis 1665 which corresponds to an axis that extends through a center of mass and/or a geometric center of the antenna 1030 and/or a location where a number of coils on one side equals a number of coils on the other and/or fractions thereof (this does not include the leads that extend to the antenna—here, the antenna begins where the coiling begins—it is also noted that these features can correspond to antenna 1020, as noted above). This axis is normal to the axis 1501, which also extends through the center of mass and/or a geometric center of the antenna 1020 and/or is the axis of winding. (Note herein that reference to antenna 1020 corresponds to a disclosure associated with the other antenna, antenna 1030, and vice versa.) The crosspoint of the axis 1665 and 1501 can be located in the vertical direction D3 from the front surface of the housing 622H (the surface that faces the lead 618). D3 can be less than or equal to or greater than 5, 6, 7, 8, 9, 10, 11, 12, 13, 14, 15, 16, 17, 18, 19, or 20 mm, or any value or range of values therebetween in 0.1 mm increments. It is also noted that while the dimension D3 has been shown as being measured from the front of the housing, in an alternate embodiment, the dimension D3 can be measured from the back of the housing (the side facing the antenna 137). It is also noted that in an exemplary embodiment, the dimension D3 can be measured from the forward most portion or the rearward most portion of the housing with respect to the frame of reference of FIG. 16.

In an exemplary embodiment, the forward most portion of the antenna 1030 (and of course, 1020) can be less than or equal to or greater than 5, 4, 3, 2, 1 or 0 mm in front of the front of the housing or any value or range of values therebetween in 0.1 mm increments or can be less than or equal to or greater than 0, 1, 2, 3, 4, 5, 6, 7, 8, 9 or 10 millimeters or any value or range of values therebetween in 0.1 mm increments in back of the front of the housing. The aforementioned values can also be the case in reverse with respect to the rearward most portion of the antenna relative to the rear of the housing.

The crosspoint of the axis 1665 and 1501 can be located in the horizontal direction from the side surface of the housing 622H that faces the antenna 1030 a distance D2, where D2 can be less than or equal to or greater than 2, 3, 4, 5, 6, 7, 8, 9, 10, 11, or 12 mm, or any value or range of values therebetween in 0.1 mm increments. It is also noted that with respect to FIG. 16, the furthest most portion of the antenna can be 11, 12, 13, 14, 15, 16, 17, 18, 19, or 20 millimeters, or any value or range of values therebetween in 0.1 mm increments from the sidewall of the housing and/or the closest most portion of the antenna can be less than or greater than or equal to 0, 1, 2, 3, 4, or 5 mm, or any value or range of values therebetween in 0.1 mm increments from the side.

Briefly, it is noted that the housings detailed herein can be housings having a length dimension L, a width dimension W, and a height dimension H, wherein L and W are variously equal to or less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 mm, or any value or range of values therebetween in 0.1 mm increments (e.g., L could be 30 mm, and W could be 24.3 mm or less than those values), and H is any value equal to or less than 20, 19, 18, 17, 16, 15, 14, 13, 12, 11, 10, 9, 8, 7, 6, 5, 4, or 3 mm, or any value or range of values therebetween in 0.1 mm increments.

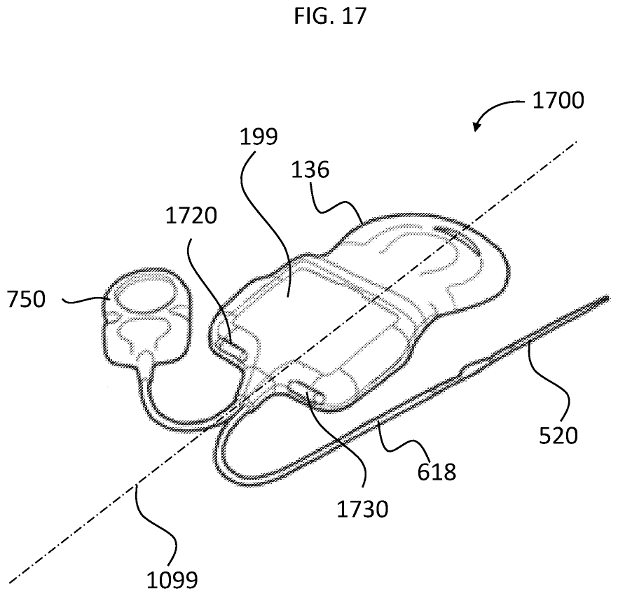

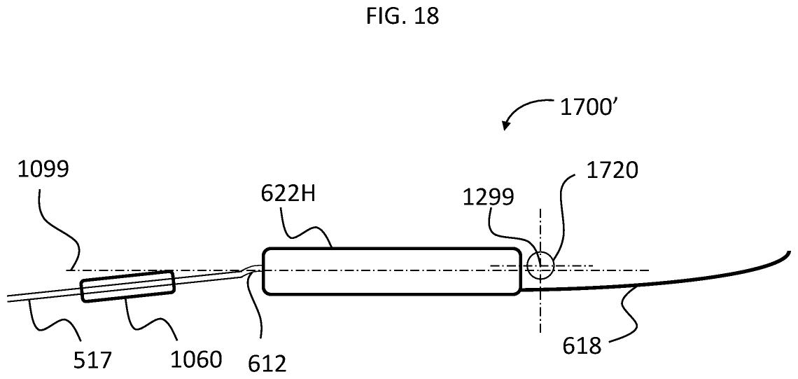

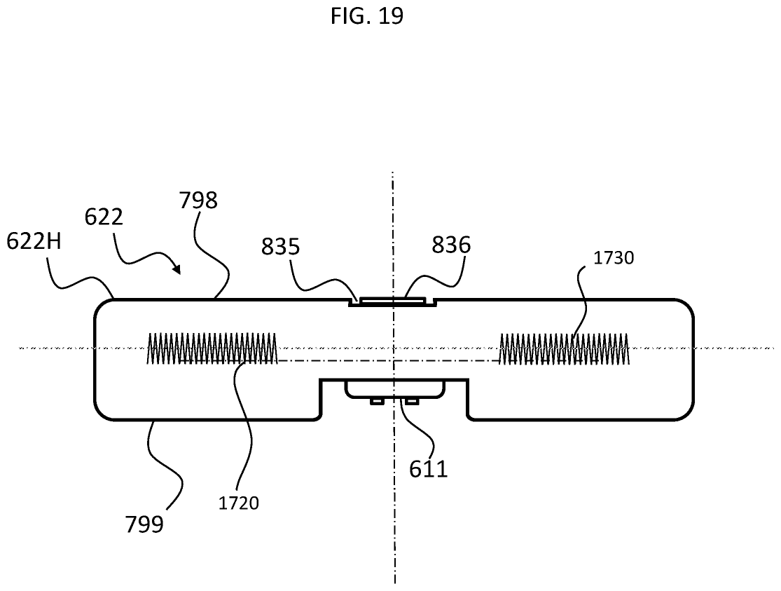

FIG. 17 depicts another exemplary implantable component 1700. Here, antennas 1720 and 1730 which can correspond to the configurations detailed above with regard to antennas 1020 and 1030 are located at the front of the housing that houses the receiver-stimulator of the cochlear implant. In this exemplary embodiment, like reference numbers correspond to the components detailed above. FIG. 18 depicts an embryonic/partial implantable component 1700′ reflecting the view of FIG. 15 above, which shows antenna 1720 on the side of housing 622H (the front side—the side that faces the cochlear implant electrode array 618). Antenna 1730 is eclipsed in this view, but is located behind the antenna 1720. FIG. 19 depicts a front view of the arrangement of FIG. 18, showing the two antennas 1720 and 1730. Accordingly, it can be seen that in an exemplary embodiment, there is a device corresponding to an implantable component of a hearing prosthesis where the second and third antennas are arranged symmetrically relative to a first plane that lies on and is parallel to a longitudinal axis of an assembly comprising the housing and the first antenna, the second and third antennas are located on the same side of the housing. Further, in an exemplary embodiment, the second and third antennas and the housing are arranged symmetrically relative to a second plane normal to the first plane and parallel to the longitudinal axis. (This is not shown in FIGS. 18 and 19—the longitudinal axis of the coil antennas/axis of winding is located closer to the top of the housing 622H than the bottom of the housing.) Briefly, it is noted that with respect to the embodiment of FIG. 19, the coils 1720 and 1730 would be located above the electrode lead assembly and on either side of the electrode lead assembly (which would extend out of the page of FIG. 19 from element 611).

In view of the arrangement of FIG. 17, it can be seen that the second and third antennas are located on a same side of the housing, which same side is an opposite side of the housing from that which the first antenna is located.

In an exemplary embodiment of the device just detailed and/or variations thereof, the second and third antennas are arranged symmetrically relative to the housing. This symmetry can be with respect to the left and right sides of the housing (where a plane parallel to the longitudinal axis 1099 bifurcates the left and right side and the second and third antennas are on the respective sides). FIG. 19 depicts this by way of example. This symmetry can also be respect to the height and/or the length of the housing (which would be seen in FIG. 11 if the axis 1299 were midway between the top and the bottom of the housing, where the antenna 1730 on the opposite side mirrors that seen in FIG. 18.

As with the embodiment of FIGS. 11 and 12, in an exemplary embodiment, of the same housing side antennas, the second and third antennas are cylindrically coiled antennas that are arranged symmetrically relative to one another.

In an exemplary embodiment, the longitudinal axes of antennas 1720 and/or 1730 are within 15, 14, 13, 12, 11, 10, 9, 8, 7, 6, 5, 4, 3, 2, 1 degrees or any value or range of values therebetween in 0.1° increments of each other as measured in any one of the frames of references of FIGS. 18, 19 and 20. As seen, in FIGS. 18 (where antenna 1730 is located on the opposite far side and mirrors antenna 1720), 19 and 20, the longitudinal axes of the coil antennas are parallel in all three frames of reference.

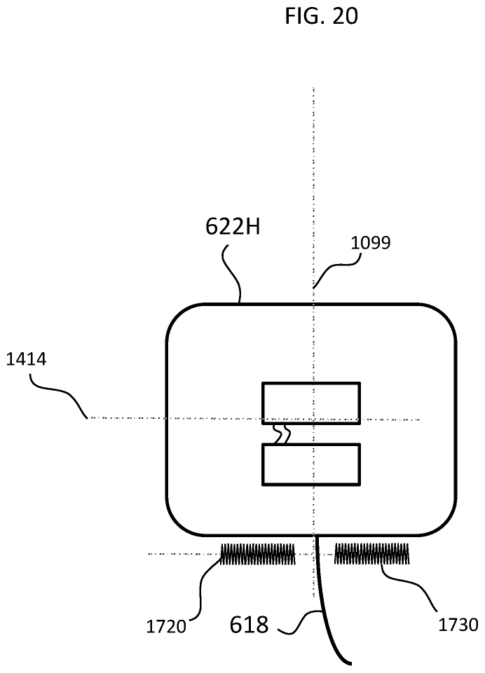

In some embodiments of the same housing side antennas, the second and third antennas are arranged symmetrically relative to a first plane that lies on and is parallel to a longitudinal axis of an assembly comprising the housing and the first antenna (or just the housing or the first antenna in some embodiments). This is seen in FIG. 17 and FIG. 20, where first antenna (coil 517, not shown), would be symmetrical about axis 1099, and the plane would be normal to the page. In some embodiments, misalignment of the two antennas relative to the plane would be less than or equal to 15, 14, 13, 12, 11, 10, 9, 8, 7, 6, 5, 4, 3, 2, or 1%, or any value or range of values therebetween in 0.1% increments on a component portion to component portion basis.

In some embodiments, misalignment of the two antennas relative to the above noted second plane vis-à-vis the same side antenna embodiment would be less than or equal to 15, 14, 13, 12, 11, 10, 9, 8, 7, 6, 5, 4, 3, 2, or 1%, or any value or range of values therebetween in 0.1% increments on a component portion to component portion basis (e.g., the height (in cylindrical terms) of antenna 1720 vs. the height (again, in cylindrical terms) of antenna 1730 above the second plane is no more or no less than 10% of a difference of the other).

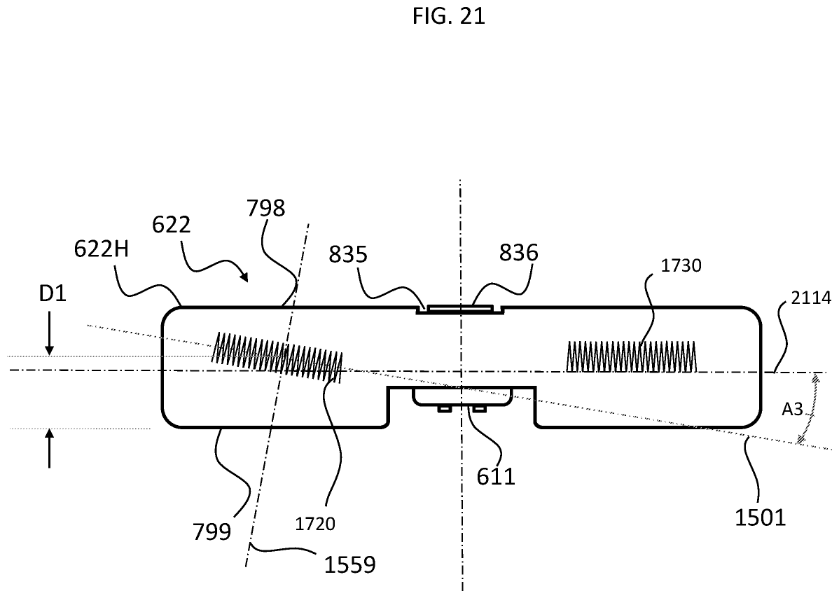

In an exemplary embodiment, the second and/or third antennas (1720 and/or 1730) are at an oblique angle relative to a plane of extension of the width and the length of the housing 622H. This can be seen in FIG. 21, which depicts the height and width of the housing (the height is the vertical direction, and the width is the horizontal direction). In an exemplary embodiment, that plane can be normal to the direction of extension of the height of the housing. In this regard, the lateral axis 2114 of the housing can represent the aforementioned plane, where the plane extends parallel to that line in and out of the page normal to the page. As seen, the longitudinal axis 1501 of the antenna 1720/the axis of winding 1501 (which can be the geometric center of the antenna and/or the center of mass of the antenna) extends at an angle A3 relative to the lateral axis of the housing 2114 (or relative to a bottom and/or a top plane of the housing 622H and/or a plane upon which the maximum diameter of the housing extends in some other embodiments). A1 can be plus or minus (the orientation shown in FIG. 21 is plus) and equal to or less than or greater than the values noted above for A3. Of course, in the embodiments where the angle is not oblique, the angle A3 can be zero.

It is noted that in some embodiments, the angle A3 of the second antenna 1720 can be different than the angle of the third antenna 1730. Any of the features detailed above with respect to antennas 1020 and/or 1030 can be applicable to antennas 1720 and/or 1730, with the appropriate variations to take into account the 90 degree change in orientation to get to the same housing side features.

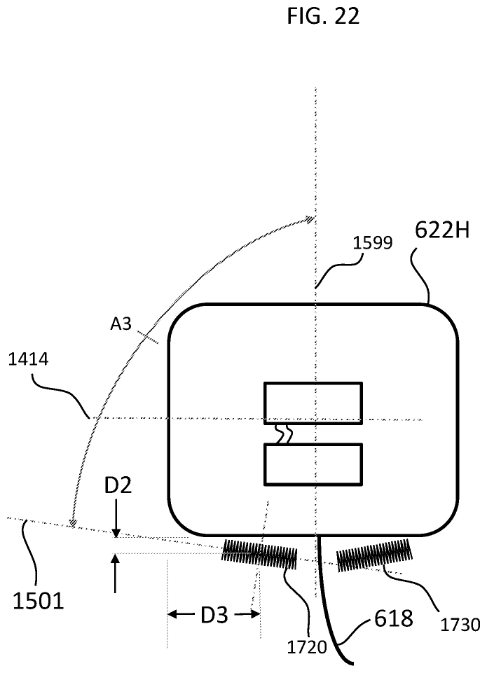

In an exemplary embodiment of the same side housing antennas, the second and/or third antennas are at an oblique angle relative to a plane of extension of a height and a length of the housing. This can be seen in FIG. 22, which shows the length and the width of the housing 622H. The aforementioned plane can be planes that lie on and are parallel to the longitudinal axis 1599 extending out of the page and normal to the page. In an exemplary embodiment, the plane can be normal to the width. Also as seen, there is an angle A3 between axis 1599 and axis 1501. A3 can be plus or minus (the orientation shown in FIG. 22 is plus) and equal to or less than or greater than 90, 98, 88, 87, 86, 85, 84, 83, 82, 81, 80, 75, 70, 65, 60, 55, 50, or 45 degrees, or any value or range of values therebetween in 0.1° increments. Also, the angle can be a generic angle which means that there is no plus or minus, it just is an angle that is oblique relative to the plane (it could be as shown in FIG. 22 or the mirror image of that about line 1599). Of course, in the embodiments where the angle is not oblique, the angle A3 can be zero.

It is noted that in some embodiments, the angle A1 and/or A3 of the second antenna can be different than the angle of the third antenna. Indeed, in an exemplary embodiment, one angle can be positive in one angle can be negative. That said, in an exemplary embodiment, the symmetrical arrangements on at least two planes can have utilitarian value.

FIGS. 21 and 22 depict some additional data points associated with some embodiments. More specifically, as can be seen, there is an axis 1559 which corresponds to an axis that extends through a center of mass and/or a geometric center of the antenna 1720 and/or a location where a number of coils on one side equals a number of coils on the other and/or fractions thereof (this does not include the leads that extend to the antenna—here, the antenna begins where the coiling begins). This axis is normal to the axis 1501, which also extends through the center of mass and/or a geometric center of the antenna 1720 and/or the axis of winding of the antenna. (Note herein that reference to antenna 1720 corresponds to a disclosure associated with the other antenna, antenna 1730, and vice versa.) The crosspoint of the axis 1559 and 1501 can be located in the vertical direction D1 from the bottom surface. In the interests of textual economy, any of the features of antenna 1020 with respect to the view of FIG. 15 can correspond to that of FIG. 21 providing that the art enables such.

Also, as seen, as with the different side embodiment, there is an axis 1665 which corresponds to that axis as detailed above. The crosspoint of the axis 1665 and 1501 can be located in the horizontal direction D3 from the side surfaces of the housing 622H. D3 can have the values noted above.

In an exemplary embodiment, the leftmost portion of the antenna 1720 (and of course, the rightmost portion of the antenna 1730) can be less than or equal to or greater than 5, 4, 3, 2, 1, or 0 mm from the side of the housing to the left (or right, with respect to the antenna 1730) or any value or range of values therebetween in 0.1 mm increments or can be less than or equal to or greater than 0, 1, 2, 3, 4, 5, 6, 7, 8, 9, or 10 millimeters, or any value or range of values therebetween in 0.1 mm increments to the right of the side of the housing. The aforementioned values can also be the case in reverse with respect to the rightward most portion of the antenna relative to the sides.

The crosspoint of the axis 1665 and 1501 can be located in the vertical direction from the front of the housing 622H/the side that faces the antenna 1720 a distance D2, where D2 can be less than or equal to or greater than 2, 3, 4, 5, 6, 7, 8, 9, 10, 11, or 12 mm, or any value or range of values therebetween in 0.1 mm increments. It is also noted that with respect to FIG. 22, the furthest most portion of the antenna can be 11, 12, 13, 14, 15, 16, 17, 18, 19, or 20 millimeters, or any value or range of values therebetween in 0.1 mm increments from the front of the housing and/or the closest most portion of the antenna can be less than or greater than or equal to 0, 1, 2, 3, 4, or 5 mm, or any value or range of values therebetween in 0.1 mm increments from the front of the housing.

Thus, in view of the above, it can be seen that in an exemplary embodiment, the second and third antennas are arranged symmetrically relative to a first plane that lies on and is parallel to a longitudinal axis of an assembly comprising the housing and the first antenna, and the second and third antennas are located on a same side of the housing. Also in view of the above, it can be seen that in some embodiments, the second and third antennas are arranged symmetrically relative to a first plane that lies on and is parallel to a longitudinal axis of an assembly comprising the housing and the first antenna or just the housing or just the first antenna (any reference herein to symmetry associated with one of these three arrangements corresponds to a disclosure of symmetry with respect to the other two arrangements). Here, the second and third antennas are located on opposite sides of the housing, and the second and third antennas and the housing are arranged symmetrically relative to a second plane normal to the first plane and normal to the longitudinal axis.

Moreover, in some embodiments, the second and third antennas are arranged symmetrically relative to a first plane that lies on and is parallel to a longitudinal axis of an assembly comprising the housing and the first antenna and the second and third antennas and the housing are arranged proximate to and on either side of a lead assembly extending from the housing to an output apparatus of the device, the output apparatus being configured to stimulate the human recipient.

Also as can be seen from the above, in some embodiments, the second and third antennas are arranged symmetrically relative to a first plane that lies on and is parallel to a longitudinal axis of an assembly comprising the housing and the first antenna and the second and third antennas and the housing are arranged unsymmetrically relative to a second plane normal to the first plane and normal to the longitudinal axis.

In an exemplary embodiment of the implantable component, the first antenna is a wide diameter inductance coil. Further, the second and third antenna are located such that, respectively, a majority of conductive componentry and/or antenna by mass thereof and/or a majority of the surface area of the antenna is located within 10, 11, 12, 13, 14, 15, 16, 17, 18, 19, 20, 21, 22, 23, 24, 25, 26, 27, 28, 29, 30, 31, 32, 33, 34, or 35 mm, or any value or range of values therebetween in 0.1 mm increments of each other. In an exemplary embodiment, at least 55, 60, 65, 70, 75, 80, 85, 90, 95, or 100%, or any value or range of values therebetween in 1% increments of the aforementioned features are located within the just detailed measurements.

In an exemplary embodiment, the coil of the first antenna 137 is at least a distance of and/or equal to a distance of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mm, or any value or range of values therebetween in 0.1 mm increments from the second antenna and/or third antenna.

Briefly, as noted above, silicone is located over the housing and the implant coil 137. In this regard, the aforementioned spatial features of the second and/or third antenna can be achieved by utilizing the silicone, or any other type of biocompatible material such as ceramic or peek material to support the second and/or third antenna. Briefly, returning back to FIGS. 10 and 17, it can be seen that the antennas are supported proximate the housing by the silicone that encases the housing. Accordingly, in an exemplary embodiment, the implanted component includes a silicone body extending about the housing, wherein the second and third antennas are supported by the silicone body.

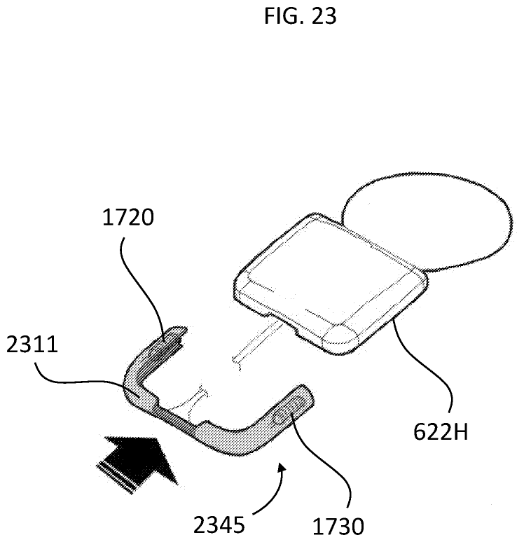

Further, in an exemplary embodiment, there is a bracket mount that is utilized to support the antennas. In an exemplary embodiment, this bracket mount is a slider bracket mount. In this regard, FIG. 23 depicts an assembly 2345 that is established at least in part by the slider bracket mount 2311 and the coils 1720 and 1730 and the associated electrical leads. Here, in an exemplary embodiment, as represented by the arrow, the slider bracket mount 2311 can be slid about the housing 622H so that the antennas 1720 and 1730 respectively are positioned on opposite sides of the housing so as to obtain the configuration of FIG. 10 above. Accordingly, in an exemplary embodiment, there is a method of manufacturing the implantable assembly which includes obtaining a housing 622H with or without a silicone body or coating thereabout (the slider bracket mount 2311 can be placed over a silicone body encasing the housing in some embodiments, while in other embodiments, the slider bracket mount 2311 is placed directly against the body of the housing (whether it is titanium or ceramic or whatever) and sliding or otherwise placing the slider bracket mount about the housing and attaching the electrical leads to a feedthrough attached to the housing. Again, it may not necessarily be the case that the bracket is slid over the housing. In an exemplary embodiment, the “arms” of the bracket are sufficiently flexible that they can be pulled outward and then permitted to spring back about the housing. In an exemplary embodiment, a sealant or some form of a piece of material or bonding material can be utilized to bond the mount 2311 to the housing and/or to the silicone over the housing. In an exemplary embodiment, the silicone can be slightly melted or otherwise heated to obtain a tacky state so that the silicone of the mount will bond to the housing and/or to the silicone over the housing. That said, in an exemplary embodiment, silicone can be placed over the assembly 2345 and the housing after the two are connected to each other, this silicone forming an outer silicone shell or body that holds everything together.

Thus, in an exemplary embodiment, the second and/or third antennas are supported by a bracket mount, such as a slider bracket mount, which is attached to the housing. That said, in an exemplary embodiment, there is no bracket per se. In an exemplary embodiment, the coils can be encased in an overall silicone body or any other type of biocompatible material such as ceramic or peek material that encases the housing and/or other components. In an exemplary embodiment, a biocompatible support structure could be utilized to position the coils relative to the housing or otherwise hold the coils relative to the housing during the silicone application process. A material that would not interfere or otherwise affect the transmissive and/or receptive properties of the coil can be utilized in at least some exemplary embodiments.

As can be understood from the above, in contrast to the large diameter inductance coil number 137, the second and/or third antennas are held to the housing in a much less flexible manner. That is, in an exemplary embodiment, owing to the silicone body 136 that supports the coil 137, the coil number 137 can be moved relative to the housing in a plane that is parallel to and lying on the longitudinal axis of the implantable assembly (e.g., with respect to the frame of reference 1C, the left side of the assembly 100 would be rotatable about the right side of the body—the left side could swing up and down). Conversely, the second and third antennas are effectively fixed relative to the housing.

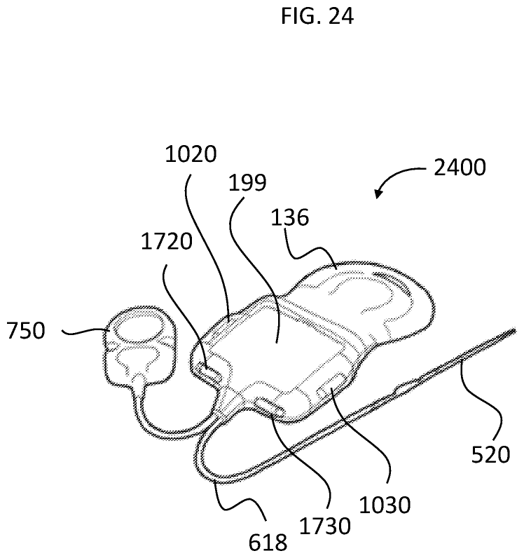

FIG. 24 presents an alternate embodiment of an implantable component, implantable component 2400, which includes the antennas 1020 on opposite sides of the housing, and the antennas 1720 and 1730 on the same side of the housing arrayed on separate sides of the lead assembly 618. In an exemplary embodiment, there can be for separate transducers for each of the antennas. In an exemplary embodiment, two of the antennas can be wired in series with each other and the remaining two antennas can be wired in series with each other. The antennas in series can utilize the same transducer (and thus there would be to transducers). In an exemplary embodiment, antennas 1730 and 1030 are wired in series and can share a common transducer, and antennas 1020 and 1720 are wired in series and can share a common transducer separate from that of the aforementioned common transducer. In an exemplary embodiment, antennas 1020 and 1030 wired in series, and can share a common transducer, and antennas 1720 and 1730 are wired in series, and can share a common transducer separate from that of the aforementioned common transducer. All of this said, a single transducer can be utilized for the sets in series, and there can be a switch or a routing arrangement that can block the signal communication from one set and/or the other set. In an exemplary embodiment, all four antennas can be wired in series, and a common transducer can be utilized for all. In an exemplary embodiment, a common transducer can be utilized for all of the antennas, but the antennas all have a home run directly to the transducer, and a switch arrangement or otherwise a routing arrangement can be utilized to variously block signal communication from one or more of the antennas to the transducer.

Two antennas can be arranged in series to a respective single transducer and two antennas can have a home run to respective transducers. Any arrangement that can enable the teachings detailed herein that have utilitarian value can be utilized in at least some exemplary embodiments.

Thus, in view of the of the above, in an exemplary embodiment, where the implantable component includes a housing hermetically housing the receiver electronics therein and where the implantable component includes a second antenna and a third antenna configured for MI radio communication with the external component, the implantable component can be configured such that the second and/or third antenna can be controllably placed in signal communication with the receiver circuitry, and in some embodiments, when one of the antennas is in signal communication with the receiver circuitry, the other antenna is controllably out of signal communication with the receiver circuitry.

As mentioned above, the second and third antennas can have utilitarian value with respect to data transmission from an external component to the implantable component. This can enable the wide diameter antenna 137 and the corresponding external antenna that is in signal communication there with to be used solely for power transfer to the implantable component, to power the circuitry of the implantable component in real time while the data is being transferred to the implantable component via receipt by the second and third antennas so that that power can be utilized to evoke a hearing percept based on that data (e.g., the data can include stimulator drive instructions that are based on captured sound captured by the microphone to the external component so that the stimulator or whatever device is implanted that will output a signal to a device that stimulates tissue (actuator, electrodes, etc.) will operate to send the signal evoke a hearing percept based on those drive instructions so that the recipient will obtain a percept of the sound). The data can be provided separately from any power transfer—this can be the case where, for example, the implanted device is a totally implantable hearing prostheses with an implantable power storage device such as a battery, and external microphones are being utilized owing to the fact that such typically have different signal-to-noise ratios and/or otherwise provide different sound capture results relative to an implantable microphone (e.g., because the implantable microphone is a layer of skin thereover, etc.), and the recipient seeks the basis for operation of the implant to be ultimately based on sound captured from an external microphone as opposed to the implantable microphone. It is also noted that in some other scenarios, data transfer can take place while the wide diameter antenna is being used to simply charge the implanted battery. That is, the onboard battery can be utilized to power the electronics of the implantable component while that battery is being recharged and while data is being provided via the second and third antenna to the implantable device.

In an exemplary embodiment, the second and third antennas are utilized for MI radio. This as distinguished from the traditional inductive signal resulting from the diameter antennas. MI radio allows data transfers using alternating magnetic waves in the magnetoquasistatic field between antenna coils up to 50 cm range. The transceivers used for MI radio communication consume very little power compared to other type of transceivers based on radiated emissions or propagating field waves.

Embodiments of the teachings detailed herein have utilitarian value with respect to enabling data transfer or otherwise signal communication from a wide variety of external component configurations for the same exact implantable component located at a variety of positions. That is, some embodiments of the implantable component as explained herein can have utilitarian value with respect to providing by semi analogy a “one size fits all” implant. Granted, this is not in terms of size (indeed, the sizes are the same—the designs are the same)—instead, this is about placement and configuration, but the analogy is apt. A single implantable component can be utilized for children and for adults (having different size heads, the latter having sometimes 2 to 3 to 4 times or more the size skull outer surface area), can be utilized for left side and for right side implantation, and can be utilized in a variety of different angles and placements within the recipient, all while permitting the same external component designs to be utilized to communicate with the implantable component via the second and/or third antennas, and also while permitting a wide variety of external component designs to be so utilized for communication with those antennas. In an exemplary embodiment, the teachings herein can enable the use of the same implant in a 1, 2, 3, 4, 5, 6, 7, 10, 15, 20, 25, 30, 35, 40, 45, 50, 55, 60, 65, 70, 75, or 80, or any value or range of values therebetween in 1 month increments 20 to 80 percentile human factors male or female born in the United States, by enabling a wide variety of placement locations, as will be detailed herein. And with respect to at least some implementations, there is no need for different implants for a left side and a right side implant—the same implant can be used for both.

By way of example only and not by way of limitation, a given implantable component can be utilized to establish the implanted portion of a transcutaneous signal link with an external component establishing the external portion of that transcutaneous link having a transmissive antenna that is fixed relative to the overall housing of the external component, and can also be utilized to establish, without moving the implanted component, a second transcutaneous signal link with an external component of a completely different form, which external component establishes the external portion of that second transcutaneous link, where a second transmissive antenna that is also fixed relative to the overall housing of that second external component is located at a different location outside the recipient. By way of example, the first link can be established utilizing a behind-the-ear device such as that detailed above in FIG. 8, and the second link can be established utilizing an ITE device such as that detailed above in FIG. 9, etc., where the antennas would be located at different locations on the outside of the recipient.

Again as noted above, the teachings detailed herein permit the establishment of a viable and otherwise high quality and/or well-defined magnetic radio inductance link between a given implant in a wide variety of designs of external components located in a wide variety of orientations.

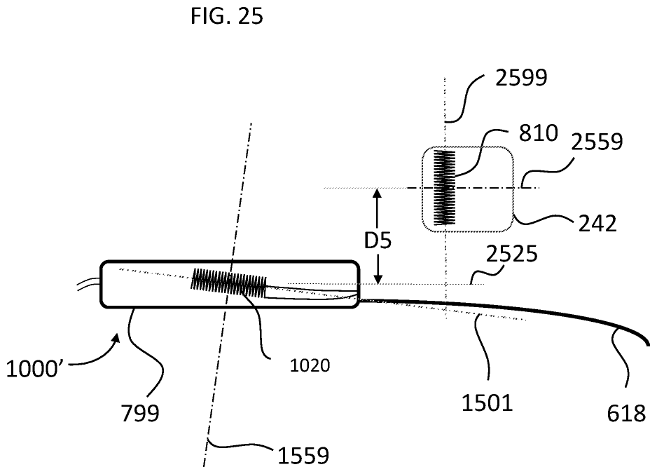

FIG. 25 depicts a view looking downward or upward with respect to a recipient's head showing a cross-section of an in implantable portion 1000′ of an implantable component of a hearing prosthesis and a cross-section of a BTE device 242, both cross sections showing the respective MI antennas. Also superimposed on this view is a distance from the aforementioned centers of the antennas (where axis 1559 and 1501 cross—axis 2599 can correspond to the latter and access 2559 can correspond to the former with respect to the geometry of the external antenna 810—the features described above with respect to the respective axes of the implantable antenna can also correspond to those of the external antenna and thus will not be elaborated on herein in the interest of textual economy).

The distance D5 between the aforementioned centers is shown in one dimension, but represents a two-dimensional distance as the axes of the coils are not on the same plane (indeed, the locations of the implanted coils will be frequently different from one person to another owing to physiology and/or implant actions). It is noted that the distance is measured from the center of the external antenna 810 along axis 2599 to a plane 2525 that is normal to that axis to the hypothetical plane that extends at a 90° angle relative to axis 2599 and extends through the axis 1501 of the antenna 1020. In an exemplary embodiment, the distance D5 can be less than, greater than, and/or equal to 5, 6, 7, 8, 9, 10, 11, 12, 13, 14, 15, 16, 17, 18, 19, 20, 21, 22, 23, 24, 25, 26, 27, 28, 29, or 30 mm, or any value or range of values therebetween in 0.1 mm increments.

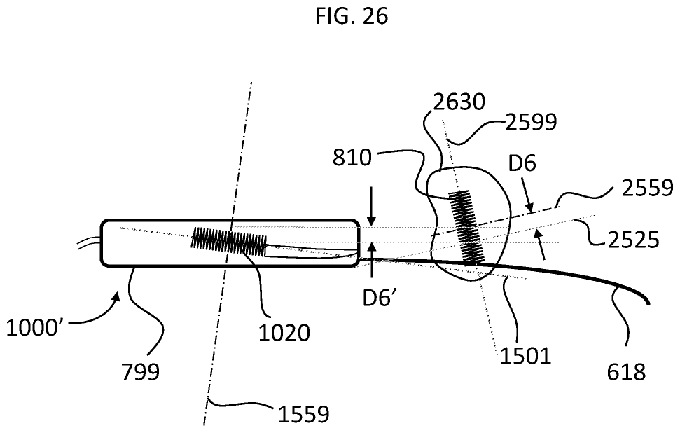

FIG. 26 depicts a view akin to that of FIG. 25, except where an ITE device 2630 supports or otherwise includes the external antenna 810 (here, the antenna 810 is located in the ear canal). In this exemplary embodiment, both antennas have longitudinal axes that are oblique relative to the ex-wife frame of reference of that view. Here, it can be seen that plane 2525 results in a measurement D6 that would correspond to a plane that is normal to the Y axis and extending through the center of the coil 1020, which measurement is made from the point at which the axis 2599 extends through that plane. This value is the same value as that which results from extending the plane 2525 to the axis 1501 as can be seen from the figure. In an exemplary embodiment, the distance D6 can be less than, greater than, and/or equal to −9, 8, 7, 6, 5, 4, 3, 2, 1, 0, 1, 2, 3, 4, 5, 6, 7, 8, 9, 10, 11, 12, 13, 14, 15, 16, 17, 18, 19 or 20 mm or any value or range of values therebetween in 0.1 mm increments. (It is noted that the frame of reference of FIG. 26 shows positive D6 value—if the plane 2559 was instead “below” plane 2525, D6 would be negative.)

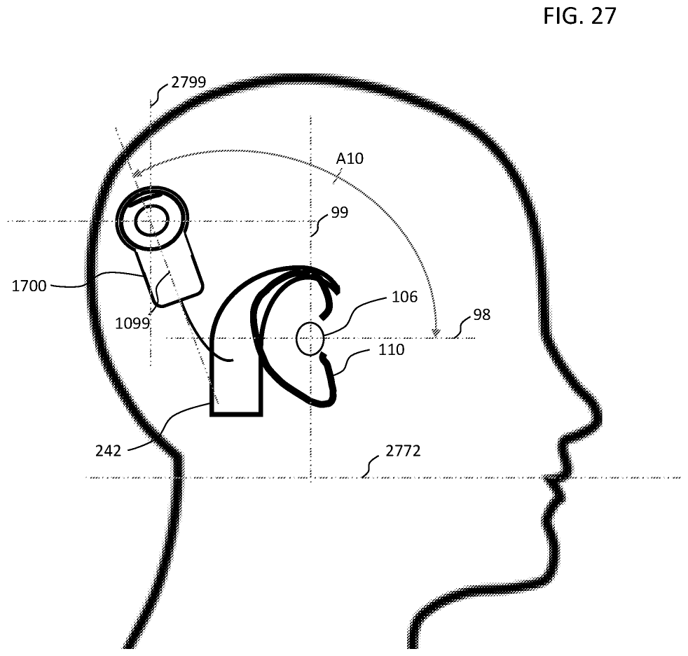

All the above said, in at least some exemplary embodiments, the pertinent distances between the centers of the antennas can be measured in a one-dimensional format via projection. FIG. 27 depicts an exemplary human head arranged perfectly on the XY axis (Y is the direction of gravity, the human standing (or at least holding his or her head) perfectly erect and perfectly to the side/a perfect silhouette)—axis 99 is the Y axis, and axis 98 is the X axis—more on this below. Shown is a plane 2772 (a cross-section of the plane, and hence a line) that extends into and out of the page which is orthogonal to the Y axis of the human associated with that figure (note that we are utilizing the terms X and Y axis with respect to the traditional manner that they are utilized on a two-dimensional figure such as those in the figures—if the frames of references were utilized in the exact same manner in three dimensions, the Y axis of FIGS. 25 and 26 would actually be the Z axis—if we kept X the same in both views). The arrangements of FIG. 26 and FIG. 27 can correspond to projections on to that plane. This would eliminate the height dimension associated with the spatial relationships. Moreover, all measurements can be taken along the Y axis (with respect to the given figures—it would actually be the Z axis in FIGS. 25 and 26 if we were utilizing a three-dimensional axis that is common to all three figures). FIG. 26 presents this concept with respect to the dimension D6′, which is measured from two planes that are normal to the Y axis and pass through the centers of the respective antennas. In an exemplary embodiment, D6′ can be any of the values just detailed for D6, and, likewise, this concept of measurement can be applied to the arrangement of FIG. 25, where the hypothetical D5′ can be any of the values just detailed for D5—indeed, the values with respect to the arrangement of FIG. 25 would be exactly the same.

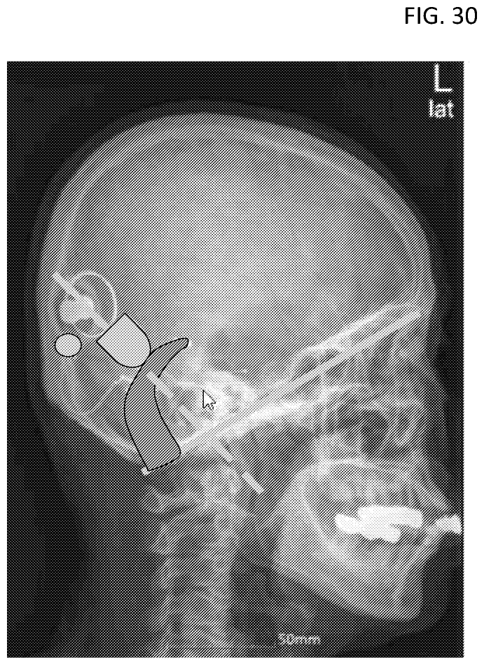

Returning back to FIG. 27, it can be seen that there is an implantable component 1700 which is representative of any of the implantable components detailed herein implanted beneath the skin of a recipient between the skull and the skin. The coordinates shown are centered about the ear canal 106 (the outer ear) and can represent a projection onto the XY plane from location of the center point of the tympanic membrane. As seen, there is a longitudinal axis 1099 of the implantable component 1700, which axis can correspond to any of the axes detailed herein, and can also correspond to an axis established by bisecting the wide diameter antenna so that equal amounts of mass and/or equal amounts of surface area and/or equal amounts of enclosed volume are located on either side of the axis. The axis can also be defined as passing through or otherwise above or below (this is a three dimensional arrangement projected onto a two-dimensional plane) the center point of the lead assembly that extends from the receiver stimulator of the implant to the output device (whether that is an actuator or an electrode array) at the location where the lead exits the housing and/or the axis can be defined as passing through the center of the housing, etc. In any event, as shown, there is an angle A10 between the longitudinal axis and the X axis 98 centered at the ear canal as noted above. In an exemplary embodiment, depending on height and/or the orientation of the implantable component, A10 can be more than or equal to 90, 95, 100, 105, 110, 115, 120, 125, 130, 135, 140, 145, 150, 155, 160, 165, 170, 175 or 180 or any value or range values therebetween in 1% increments. Depending on the height and/or angle, the positioning of the implantable component can be considered to be located in an “up” position or a “back” position (with “back” positions having a value of A10 further away from 90 degrees than the “up” positions). Note that there can be scenarios where the placement is both up and back—however, there will be one dominant over the other, and that will define the up or back. In an exemplary embodiment, if the longitudinal axis of the implant (e.g., the axis bifurcating the coil and passing through/over the lead where the lead contacts the housing), extends through the boundaries of the skull border axis, it is up, otherwise, it is back. This is seen in FIG. 30, where the solid line is the axis (and the ends are the boundaries) of the skull border axis, and the dashed line is the longitudinal axis of the implant.

Further as can be seen, there is a cross-hair 2799 that is centered about the geometric center of the coil/wide diameter antenna of the implant/the antenna utilized for power. The crosshairs are parallel to the X and Y axis. In an exemplary embodiment, the parallel axes are less than, greater than or equal to 1, 1.25, 1.5, 1.75, 2, 2.25, 2.5, 2.75, 3, 3.25, 3.5, 3.75, 4, 4.25, 4.5, 4.75, 5, 5.25, 5.5, 5.75 or 6 inches or any value or range of values therebetween in inch increments from each other. Also, with respect to the horizontal axes, the values could be less than or equal to −1, −0.75, −0.5, −0.25 or 0 inches or any value or range of values therebetween in 0.01 increments from each other in at least some exemplary embodiments. Again, all of these values are a result of projection onto the frame of reference seen in FIG. 27.

In view of the teachings detailed herein with respect to the MI radio antennas, it can be seen that the arrangement of the implant as disclosed herein can enable at least adequately defined links to be established between the implanted antenna(s) and the external antenna(s) over a wide range of placements and orientations of the implanted component. It is noted that in at least some exemplary scenarios of use, not all of the aforementioned values may work for all antenna placements and/or antenna types. Still, a great many of them will be applicable and otherwise provide a functioning link that can enable the prostheses to be operated in a utilitarian manner. More specifically, it will be understood that in embodiments where the external component utilizes an ITE device will generally require the implanted antennas to be closer to the ear canal than that which would be the case with respect to embodiments that utilize the BTE device as the external component containing the MI radio antenna. Thus, some of the aforementioned values would not necessarily be applicable to the former while those values would be applicable to the latter. Still, the point is that the teachings detailed herein enable a wide range of placements with respect to location and orientation of the implant relative to that which would be the case in the absence of the teachings detailed herein.

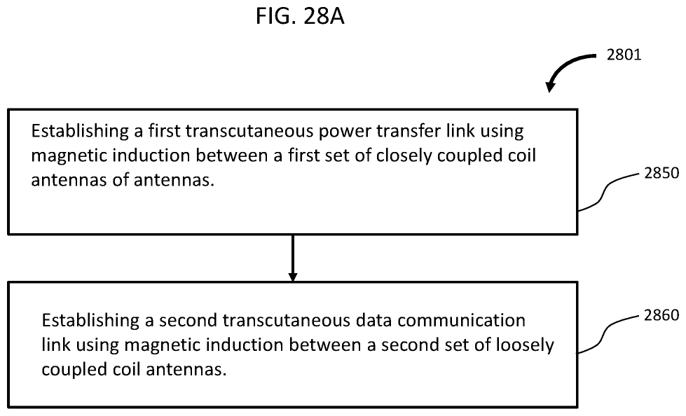

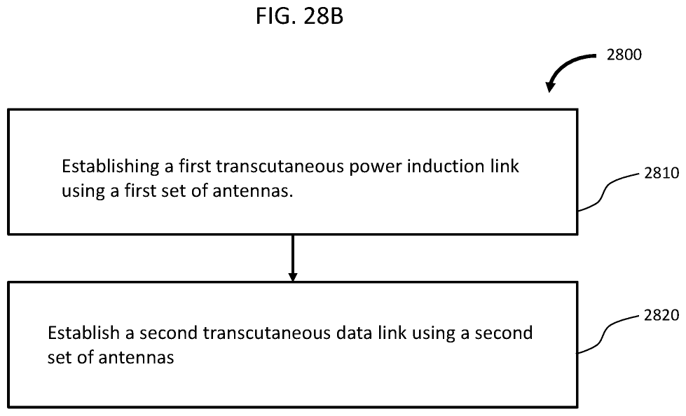

In view of the above, it is noted that there are embodiments that include methods. Referring now to FIG. 28A, there is an exemplary flowchart for a method 2801, which includes method action 2850, which includes establishing a first transcutaneous power transfer link using magnetic induction between a first set of closely coupled coil antennas of antennas. In an exemplary embodiment, the coil antennas have a clearance less than 20, 19, 18, 17, 16, 15, 14, 13, 12, 11 or 10 mm or any value or range of values therebetween in 0.1 mm increments. The clearance is mainly defined by the thickness of the skinflap between the implant and external power coil antenna. This can be executed utilizing the wide diameter antennas detailed herein, such as antenna 137 and the corresponding antennas and the associated antenna of the external component. Method 2801 further includes method action 2860, which includes establishing a second transcutaneous data communication link using magnetic induction between a second set of loosely coupled coil antennas. In an exemplary embodiment, the coil antennas have a clearance greater than 5, 6, 7, 8, 9, 10, 11, 12, 13, 14, 16, 17, 18, 19 or 20 mm.

It is noted that in this embodiment, the first and second links are established simultaneously.

In an exemplary embodiment, this set of antennas can include one or more of the implanted antennas for the MI radio system detailed herein (e.g., the narrow diameter coils), and one or more of the external antennas for the MI radio system detailed herein.

In an exemplary embodiment of method 2801, the first and second transcutaneous links are established by the same or respective different external components of a prosthetic hearing implant system and the same implanted component of the prosthetic hearing implant implanted in a human recipient. Also, in method 2801, in an exemplary embodiment thereof, at least one of: (1) the second transcutaneous link is established using antenna diversity via the implantable component, or (2) the second transcutaneous link is established using antenna and receiver diversity by the implantable component.

In an exemplary embodiment, the power transfer link may also establishe a communication link by altering power level or implant load (which can be a third communication link). This can be used to provide data for the purpose of power transfer control. In an exemplary embodiment, the second transcutaneous link is established using antenna and receiver diversity by the implantable component. That is, there can be two coil antennas and two transceivers. In an exemplary embodiment, the second transcutaneous link is established using antenna diversity via the implantable component. This can be two coil antennas and a single transceiver or two transceivers.

An exemplary embodiment includes a method of selecting a given antenna from among the implanted antennas based on a link quality. In this regard, in an exemplary embodiment, there is a method, comprising establishing a transcutaneous or subcutaneous data communication link using magnetic induction with an implanted component including magnetic induction radio communication circuitry connected to an implantable antenna arrangement of the implantable component comprising at least two coil antennas. This implantable component can be any of the implants detailed herein having two or more coil antennas, for example. In an exemplary embodiment, the method includes selecting one coil antenna of the at least two coil antennas for connection to the magnetic induction radio communication circuitry based on data based on a link quality associated with the selected one coil antenna, which selected antenna is used to establish the communication link. By way of example only and not by way limitation, depending on the positioning of the implant relative to the external component, for example, or other implantable component for example, the antenna that has the best link quality, or otherwise the link quality that has the most utilitarian value, can be selected. This provides versatility with respect to the positioning of the various components. In an exemplary embodiment, this can have utilitarian value with respect to providing a wider range of implant scenarios/positions relative to that which would otherwise be the case.

In an exemplary embodiment, link quality can be identified by verifying and/or comparing a link for each implant coil antenna. Link quality verification can be done, for example, by signal level measurement(s) and/or signal-to-noise measurement(s) bit error and/or packet error measurement(s), and comparing the various measurements. In an exemplary embodiment, the implantable component and/or the external component is configured to perform one or more or all of these measurements to evaluate the link quality, and includes logic circuitry/programming, to select a given coil.

In an exemplary embodiment, the action of selecting is executed by comparing a link quality associated with one or more antennas of the at least two coil antennas other than the selected antenna with the link quality associated with the selected antenna. In an exemplary embodiment, this is executed all of the data communication antennas. Thus, with respect to the embodiments where there are two coil antennas as detailed herein that are utilized for radio communication, this would be done for both antennas, and a link quality that is deemed to be more utilitarian would result in the antenna being selected that corresponds to such. If there were three or more antennas, this can be done three times.

That said, it is also noted that in some embodiments, the action of selecting the antenna does not necessarily require a comparison. If the link quality is acceptable or otherwise utilitarian, that can be the antenna selected, and it may not necessarily be needed to evaluate the link quality of the other antennas. In an exemplary embodiment, if the link quality is not sufficiently utilitarian, or it is believed that the link quality might be better with another antenna, then the link quality for the other antenna can be determined.

In an exemplary embodiment of this method, the data communication link is between the selected one coil antenna and another antenna of another component separate from the implantable component. This can be the external component, such as the BTE device detailed above, or can be another implantable component. Further, in this exemplary embodiment, the link qualities respectively correspond to the links between that another antenna and the respective antennas of the at least two coil antennas. In an exemplary embodiment, the data communication link is between the selected one coil antenna and another antenna of another component separate from the implantable component, and the link qualities correspond to the link between respective different antennas of the another component and the respective antennas of the at least two coil antennas.

In view of the above, some embodiments include electrically decoupling one or more of the at least two coil antennas other than the selected one coil antenna from at least some of the circuitry, based on an evaluation of link quality, and electrically coupling the selected one coil antenna to at least some of the circuitry. The idea here is that for a shared transceiver, for example, the selected antenna is in signal communication with that transceiver, and the antenna that will not be used is no longer in signal communication with that transceiver. That said, where embodiments use dedicated transceivers, the method can include deactivating a receiver (which can be a transceiver—the transceiver has a receiver) electrically coupled to one or more of the at least two coil antennas other than the selected one coil antenna based on an evaluation of link quality and activating a receiver electrically coupled to the selected one coil antenna, wherein the receivers are part of the circuitry.

In some embodiments, the action of selecting is executed after the implantable component has been implanted in a recipient. In this regard, at least some exemplary embodiments of utilitarian value with respect to enabling the selection and thus the activation of a given antenna after implantation. In an exemplary embodiment, the selection can be controlled via a data signal provided from the external component to the implantable component (or other component in signal communication with the component at issue). In an exemplary embodiment, the selection can be controlled utilizing the wide area antenna for example, in the event that the data signals per se are not readily usable because the selection has not yet taken place. Still, in at least some exemplary embodiments, the coil antennas, at least one of them, are utilized to provide the control signal or otherwise to control the implantable component to “deactivate” a given antenna and/or a given transceiver. Still further, in an exemplary embodiment, the implantable component can be configured to automatically deactivate or otherwise remove the signal communication between a transceiver and a given coil antenna. Indeed, in an exemplary embodiment, the implantable component can include logic circuitry or the like that can evaluate the link quality autonomously.

The teachings detailed herein include a computer readable medium having recorded there on, machine-readable instructions that can enable any one or more of the functions and/or methods detailed herein. In particular, the implantable component can include a computer readable medium to evaluate a link quality and select the given coil.

As an aside, it is noted that the data transfer and the power transfer utilizing the separate antennas can be executed simultaneously over at least a period of 0.25, 0.5, 0.75, 1, 1.5, 2, 2.5, 3, 3.5, 4, 5, 6, 7, 8, 9 or 10 hours or longer.

Referring now to FIG. 28B, there is an exemplary flowchart for a method 2800, which includes method action 2810, which includes establishing a first transcutaneous power link using a first set of antennas. This can be executed utilizing the wide diameter antennas detailed herein, such as antenna 137 and the corresponding antennas and the associated antenna of the external component. Method 2800 further includes method action 2810, which includes establishing a second transcutaneous data link using a second set of antennas. In an exemplary embodiment, this set of antennas can include one or more of the implanted antennas for the MI radio system detailed herein (e.g., the narrow diameter coils), and one or more of the external antennas for the MI radio system detailed herein. In this exemplary embodiment, there is no power that is transmitted over the second transcutaneous inductance link (power used to power the implanted components) and there is no data transmitted over the first transcutaneous inductance link. That said, in some embodiments, this may not necessarily be the case. There could be utilitarian value, for example, in transmitting data over the first link, which data is not as critical as the data transmitted over the second link. In an exemplary embodiment, a high Q value can exist for the first link relative to the second link. In an exemplary embodiment where data is transmitted over the first link, the data could be limited to data that would not be affected by issues associated with that high Q value. In an exemplary embodiment, the Q value of the first link is at least and/or equal to 10, 15, 20, 30, 35, 40, 45, 50, 55, 60, 65, 70, 75, 80, 90, 100, 110, 120, 130, 140, 150, 160, 170, 180, 190, 200, 225, 250, 275 or 300% or any value or range of values therebetween in 1% increments greater than the Q value of the second link. Moreover, with respect to this method, the aforementioned Q values are maintained for a period of time that is at least 5, 10, 20, 25, 30, 35, 40, 45, 50, 55, 60, 70, 80, 90, 100, 125, 150, 175, 200, 300, 400, 500, 600, 700, 800, 900 or 1000 seconds or any value or range of values therebetween in one second increments, for at least 80, 85, 90, 95 or 100% of those times. In an exemplary embodiment, the Q value of the power transmission link is at least or equal to 60, 65, 70, 75, 80, 85, 90, 95, 100, 105, 110, 115, 120, 125, 130 or more or any value or range of values in 1 increments.

In the exemplary embodiment of the method 2800 under discussion, the first and second transcutaneous links are established by the same or respective different external components of a prosthetic hearing implant system (e.g., the assembly of 242 of FIG. 2C also including the antenna 810 in an embodiment where the same component is used, and an over-the-ear battery charging device completely self-contained without contact with the pinna and a BTE device without a headpiece containing an antenna 810 (or an ITE device with such an antenna) in embodiments where the different external components of the system are utilized) and the same implanted component of the prosthetic hearing implant implanted in a human recipient (e.g., any of the implantable components detailed herein).

In an exemplary embodiment of the aforementioned method, the data link and the power link are RF frames or bursts within a TDMA scheme ink. In an exemplary embodiment of the aforementioned method, the data link and the power link are established without a combined link and without a split link. In an exemplary embodiment, the data link is a pure data link, and the power link is a pure power link. Still, as noted above, in some embodiments, there can be limited data associated with the power link, such as where the data it is course data/data that is low data content (which might be to activate or deactivate a switch, which data is data that is very unlikely to be affected by a high Q value link) versus data that is high data content, such as the data corresponding to data based on captured sound, which data is utilized to evoke a hearing percept.

In an exemplary embodiment, the aforementioned power links and/or the aforementioned data links are maintained for at least and/or equal to 30, 45, 60, 90, 120, 150, 180, 200, 250, 300, 350, 400, 450 or 500 minutes straight or any value or range of values therebetween in one minute increments.

Embodiments according to at least some of the teachings detailed herein contemplate the utilization of a BTE device to establish the second link and then subsequently the utilization of an ITE device to establish a subsequent link utilizing an ITE device to replace that second link. In at least some exemplary sub scenarios of the scenario, the location of the antenna of the external component that transmits the data to the implanted antenna will change relative to that which was the case for the subsequent link. This owing to the fact that the physical arrangements of the BTE device are substantially different from that of the ITE device. Accordingly, in an exemplary embodiment of method 2800, the second set of antennas is established by a first antenna external to an ear canal of the recipient and a second antenna implanted in the recipient, and the method further comprises, after establishing the second link, breaking the second link and establishing a third transcutaneous data link using a third antenna in the ear canal of the recipient and the second antenna, the second antenna being at the at the location where the second antenna was when the second link was established. In an exemplary embodiment, precedent to the establishment of the third link, the first link is also broken and then reestablished, utilizing the first set of antennas where the antennas of that first set are located where they were when the first link was established in method action 2810. In an exemplary embodiment, the reestablishment of the first link is executed within 1, 2, 3, 4, 5, 6, 8, 9 or 10 minutes or any value or range of values therebetween in one minute increments of the establishment of the third link.

In an exemplary embodiment, a distance from the location where the second antenna was located to the distance where the third antenna is located is greater than or equal to 2, 3, 4, 5, 6, 7, 8, 9 or 10 mm or any value or range of values therebetween in 1 mm increments. In an exemplary embodiment.

It is noted that in an exemplary embodiment, the third link could be established before the second link. That is, and in the ear device could be utilized for the utilization of the BTE device that was utilized to establish the second link. That is, in an embodiment, the aforementioned order of actions could be reversed vis-à-vis the second link and the third link.

In view of the above, it can be seen that in at least some exemplary embodiments, with respect to the external antenna establishing the second and third links, the antenna establishing the second link is shadowed at least in part by the pinna of a human, whereas the antenna establishing the third link is located such that the pinna does not shadow the antenna. Also, with respect to the locations of the antennas, in at least some exemplary embodiments, the distance between the antenna utilized in the BTE device and the implanted antenna will be less than the distance between the antenna utilized in the ITE device and the implanted antenna. In an exemplary embodiment, the differences in distance can be at least or equal to 15, 20, 25, 30, 35, 40, 45, 50, 60, 70, 80, 90, 100, 110, 120, 130, 140, 150, 160, 170, 180, 190 or 200 percent or more or any value or range of values therebetween in 1% increments where the closest distance is the baseline from which these percentages are calculated.

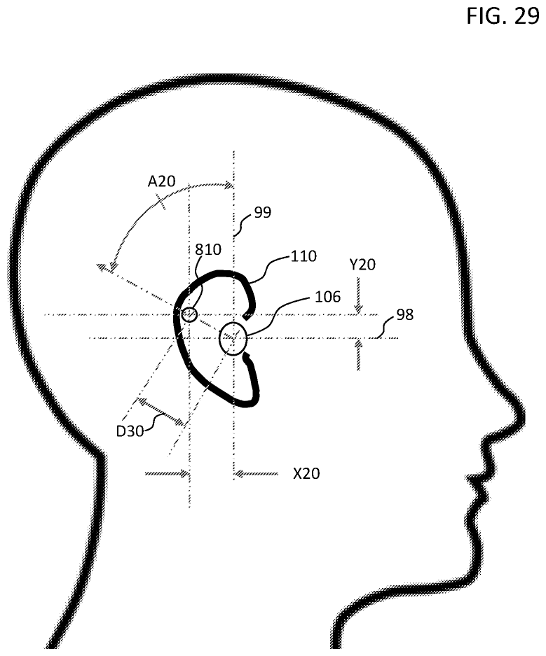

With respect to FIG. 29, there is a Cartesian coordinate system presented that is centered about the ear canal 106 (outer canal) of the recipient, and also a polar coordinate system centered about the ear canal 106. As can be seen, the Cartesian coordinate system is established by a vertical line 99 and a horizontal line 98 centered at the center of the ear canal 106 (the origin is the center of the ear canal 106, and has the coordinates 0, 0). The polar coordinate system is established utilizing the vertical line 99, the angle A1 and the distance D30 (the origin is the center of the ear canal). With respect to the schematic of FIG. 29, the plane of the schematic is taken at the location that passes through the outermost portion of the ear canal that has skin on all sides lying on a plane that is parallel to the orientation shown in FIG. 29 (for example, if the plane was located say a few millimeters closer to the viewer, the shape of the ear canal would look like a C-shape instead of a closed oval). This is also the case, in some embodiments, for FIG. 27. All shapes are superimposed outlines that would be projected on that plane when viewed with the orientation of FIG. 29, but it is noted that the shapes may not be exactly as they appear.

Shown in FIG. 29 is a diagram depicting an exemplary location of a coil antenna 810 of the second link, although the frame of reference of FIG. 29 can also be utilized and will also be utilized to describe the frame of reference for the coil antenna 810 that establishes the third link (the link established by an ITE device). We utilize the same shape in FIG. 29 to represent both of these features owing to the fact that FIG. 29 is presenting spatial relationships. FIG. 29 presents dimensions with respect to center points of features that are projected onto the plane as defined above. In this regard, the centerpoint of antenna 810 is shown with respect to the crosshairs. This centerpoint is projected onto the plane. In the embodiment depicted in FIG. 810, the longitudinal axis of antenna 810 of a BTE device extends normal to the plane of that figure. This can also be the case with respect to an antenna 810 of an ITE device that is located in the ear canal but also the angle could also be different. In an exemplary embodiment, the longitudinal axes of the antennas of the external component establishing the second and/or third link is, with respect to the frame of reference of FIG. 29, can be less than or equal to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or 60 degrees or any value or range of values there between in 1 degree increments from the direction normal of that page.

In an exemplary embodiment, X20 can be equal to or less than 0 to 45 mm or any value or range of values therebetween in 0.1 mm increments (e.g., 6, 8.83, 7.1 to 22 mm). X20 can also be greater than or equal to −20, −10, or 0 mm, or any value or range of values therebetween in 0.1 mm increments. (Note that measurements to the right of line 99 are negative values.) In an exemplary embodiment, Y20 can be equal to or less than 0 to 30 mm, or any value or range of values therebetween in 0.1 mm increments (e.g., 2.2, 4.4, 3.4 to 29.3, etc.). Y20 can also be greater than or equal to −20, −10 or 0 mm, or any value or range of values therebetween in 0.05 inch increments (Note that measurements below line 98 are negative values). In an exemplary embodiment, D30 can be equal to or less than 5 to 45 mm, or any value or range of values therebetween in 0.1 mm increments. In an exemplary embodiment, A20 can be equal to or less than 0, 10, 15, 20, 25, 30, 35, 40, 45, 50, 55, 60, 65, 75, 80, 85, 90, 95, 100, 105, 110, 115, 120, 125, 130, 140, 150, 160, 170, 180, 190, 200, 210, 220 or 230 degrees, or any value or range of values in 1 degree increments. A20 can also be greater than −130, −120, −110, −100, −90, −80, −70, −60, −50, −40, −30, −25, −20, −15, −10, −5 or 0 degrees, or any value or range of values in 1 degree increments.

To be clear, the various values are for various arrangements of placements of the antenna of the external component that establishes the second or third link. Some values would not be applicable for one versus the other. Also, embodiments also contemplate other devices that can enable these values to be met, such as a head set for a headband or the like. Accordingly, these values are applicable when the art enable such depending on the type of external component.

It is noted that embodiments can enable the establishment of the various links having sufficient quality and otherwise being sufficiently well-defined so that data can be transmitted over both links, even when the orientations of the various antennas are different relative to one link versus another. In an exemplary embodiment, the teachings herein can eliminate or otherwise reduce the occurrences are the likely occurrences of so-called dead spots or dead zones. Additional details of this will be described below, but in an exemplary embodiment, it is noted that, all other things being equal, with respect to the methods detailed herein, a strength of the third link has a value that is within 1, 2, 3, 4, 5, 6, 7, 8, 9, 10, 15, 20, 25, 30, 35, 40, 45 or 50% or any value or range of values therebetween in 1% increments of a value of a strength of the second link for an equal amount of power consumption.

Briefly, consistent with the teachings above, in an exemplary embodiment, the second set of antennas that are utilized to establish the second link comprise narrow diameter coil inductance antennas and the first set of antennas comprise wide diameter coil inductance antennas. Also, in an exemplary embodiment, the first power link is an inductance link, and the second power link is an MI radio link. In an exemplary embodiment, second link and/or the third link operates at a frequency that is at least half that of the first link or at least twice that of the first link. In an exemplary embodiment, the first link operates between 3 and 7 MHz.

In an exemplary embodiment, the first set of antennas includes a first coil external to the recipient and a second coil implanted in the recipient, which coils have respective longitudinal axes that are at least generally aligned with each other, and the second set of antennas includes a third coil external to the recipient and a fourth coil implanted in the recipient, which coils have respective longitudinal axes that are offset by at least and/or equal to 10, 15, 20, 25, 30, 35, 40, 45, 50, 55 or 60 degrees or any value or range of values therebetween in at least one frame of reference. In an exemplary embodiment, the offset can be seen in at least two frames of reference, where the frames of reference are orthogonal to one another. In an exemplary embodiment, the offset of the axes of the first antennas can be less than or equal to 10, 9, 8, 7, 6, 5, 4, 3, 2, 1 or zero degrees or any value or range of values therebetween in 0.1° increments.

In at least some exemplary embodiments, the first set of antennas includes a first coil external to the recipient and a second coil implanted in the recipient, which coils have respective longitudinal axes that have a closest approach to each other of a first value. In an exemplary embodiment, the second set of antennas includes a third coil external to the recipient and a fourth coil implanted in the recipient, which coils have respective longitudinal axes that have a closet approach to each other of a second value, which second value is at least and/or equal to 1, 1.5, 2, 2.5, 3, 3.5, 4, 4.5 or 5 times or any value or range of values therebetween in 0.1 increments that of the first value. In an exemplary embodiment the first value is less than or equal to 0.5, 1, 1.5, 2, 2.5, 3, 3.5, 4, 4.5, 5, 5.5, 6, 6.5 or 7 millimeters or any value or range of values therebetween in 0.1 millimeter increments.

In an exemplary embodiment, instead of the longitudinal axes being the basis for the measurements, the aforementioned closest approach can be a portion of the antenna (which does not include leads thereto as used herein).

Exemplary embodiments include a system for magnetic induction communication between an implantable component and an external component. The implantable component can correspond to implantable component 1000 or any of the other implantable components detailed herein meeting the following features. The implantable component can include magnetic induction radio communication circuitry connected to an implantable antenna arrangement of the implantable component, the implantable antenna arrangement comprising at least two coil antennas for radio communication. This magnetic induction radio communication circuitry can be a receiver and/or a transceiver that multiplexes or otherwise routes signals received from the various antennas.

The system further includes an external component of the hearing prostheses. This external component can correspond to the BTE device detailed above with the ITE device, or any other external component that meets the following features. This external component can include magnetic induction radio circuitry connected to a coil antenna of the external component. The radio circuitry can correspond to one or more separate transmitters and/or transceivers or an integrated transmitter and/or transceiver that multiplexes or otherwise routes signals that are to be sent to the various antennas.

In an exemplary embodiment of the system, the system is configured so that when the implantable antenna arrangement of the implantable component is implanted between a skull and skin of a human (e.g., above the mastoid bone) and the external component is worn on the head of the component during normal use, the magnetic induction communication link between the external and the implantable component is active and effectively operating.

In an exemplary embodiment of the system, the magnetic induction radio communication circuitry includes at least two separate transceivers and the respective coils of the implantable antenna arrangement are electrically connected to respective transceivers of the at least two separate transceivers. In an exemplary embodiment, the aforementioned magnetic induction radio communication circuitry of the system includes at least one transceiver and the implantable component is configured so that respective coils of the implantable antenna arrangement can be electrically connected to and electrically disconnected from the at least one transceiver. This can be done with switch devices, etc.

In some embodiments of the system, the magnetic induction radio communication circuitry detailed above includes at least one transceiver and the implantable component includes switching circuitry configured to place respective coils of the implantable antenna arrangement into electrical connection and electrical disconnection with the at least one transceiver. Also, in some embodiments of the system, the implantable coil antennas reside at separate locations and have respective longitudinal axes that are parallel to each other or are quasi-aligned with each other. In some embodiments, the coil antennas are outside the housing that contains the communication circuitry, but in some embodiments, the coil antennas are inside, such as where the housing is transparent to the signals (e.g., a ceramic housing of a given design enabling such).

Exemplary embodiments include a system comprising an implantable component of a hearing prosthesis, the implantable component including receiver circuitry in signal communication with two separate antennas. Implantable component can correspond to the implantable component 1000 or any of the other implantable components detailed herein meeting the following features. In an exemplary embodiment, the receiver circuitry can correspond to multiple receivers and/or transceivers or an integrated receiver that multiplexes or otherwise routes signals received from the various antennas. It is briefly noted that as used herein, the terms receiver and transmitter would also be met by a transceiver.

Further, the system includes an external component of the hearing prostheses. This external component can correspond to the BTE device detailed above with the ITE device, or any other external component that meets the following features. This external component can include transmitter circuitry in signal communication with two separate antennas. This transmitter circuitry can correspond to one or more separate transmitters, or integrated transmitter that multiplexes or otherwise routes signals that are to be sent to the various antennas. Again, transmitter circuitry includes the circuitry of a transceiver.

In exemplary embodiments of the system, the system is configured such that a first antenna of the implantable component receives power via an inductive transcutaneous link from a first antenna of the external component. In some embodiments, this can be the antenna 130 of FIG. 1A or the antenna 228 of FIG. 2C. Further, the system is configured such that a second antenna of the implantable component receives data via a magnetic inductance radio link from a second antenna of the external component. This can correspond to antenna 810 detailed above in some embodiments.

Moreover, the system is configured so that when the first antenna of the implantable component is implanted between a skull and skin of a human behind and/or above a pinna of the person and the external component is worn on the head of the component during normal use and both links are active and effectively operating: (i) the first antenna of the external component has a longitudinal axis that is generally aligned with a longitudinal axis of the first antenna of the implantable component and (ii) the second antenna of the external component has a longitudinal axis that is nonaligned with a longitudinal axis of the second antenna of the implantable component. In an exemplary embodiment, the axes of the second antennas are at an effective oblique angle relative to one another. Some additional details of effective relative angles will be described below. It is briefly noted that in some embodiments, the axes may be oblique relative to one another but effective communication between the two antennas may not necessarily be achievable owing to dead zones.

In at least some exemplary embodiments, the system is configured so that when the first antenna of the implantable component is implanted between a skull and skin of a human behind and/or above a pinna of the human and the external component is worn on the head of the human during normal use and both links are active and effectively operating, the first antenna of the external component is at least and/or equal to 15, 16, 17, 18, 18, 20, 21, 22, 23, 24, 25, 26, 27, 28, 29, 30, 31, 32, 33, 34, 35, 36, 37, 38, 39 or 40 mm or any value or range of values therebetween in 1 mm increments from the second antenna of the external component.

It is briefly noted that any reference to a person or human herein corresponds to a disclosure of a 50 percentile male or a 50 percentile female of 40 years of age unless otherwise noted. That does not mean that any reference to a person or human means that that is the person or human that corresponds to such. In embodiments where there is implantation into a human, that human can be a child or an adult unless otherwise specified. The reference to the 50 percentile person is a reference for disclosure purposes only.

Another exemplary method according to the teachings detailed herein entails utilizing the same implantable component at a variety of orientations and sides of the head implantation arrangements over a number of different patients/recipients. In this regard, the teachings detailed herein can be such that a one-size-fits-all implant can be provided. Briefly, owing to the arrangements of the antennas, the implantable component can be implanted with less attention paid to the location of the antennas relative to that which would otherwise be the case. Accordingly, in an exemplary method, there is the action of implanting, on a right side of a first human, a first implantable component of a hearing prosthesis including a wide diameter antenna so that the wide diameter antenna is implanted behind or above a pinna of the first human. This method also includes implanting, on a right side of a second human, a second implantable component of a hearing prosthesis including a wide diameter antenna so that the wide diameter antenna is implanted behind or above a pinna of the second human. In an exemplary embodiment, these two method actions of implanting occur within a week or a month of each other, and are executed by the same surgeon. More specifics regarding these methods of implantation include the specifics that the first and second implantable components are the same design. By way of example only and not by way of limitation the locations of the antennas are all at the same location. This as opposed to a design where, for example, there were only antennas on one side of the housing for the first person and antennas on the other side of the housing for the second person. Further, the orientations of the implantable components after implantation are substantially different. In an exemplary embodiment, the orientations can be any of the orientations detailed herein (some of which may not be substantially different of course). In some exemplary embodiments, the design of the implantable components is a design that receives power via the wide diameter antenna and data via an antenna system separate from that associated with the wide diameter antenna including at least two antennas spaced away from the wide diameter antenna.

Further, in some embodiments, a link quality between the antenna system of the first implantable component and an antenna of an external component within a housing of a spine of a BTE device will be effective to communicate data to the antenna system of the first implant so that an effective hearing percept can be evoked in the first human, and a link quality between the antenna system of the second implantable component and the antenna of the external component within the housing of the spine of the BTE device will be effective to communicate data to the antenna system of the second implant so that an effective hearing percept can be evoked in the second human when the external component is worn by the second person in the same way as worn by the first person, all other things being equal. It is noted that this does not mean that the method requires that the utilization of the same BTE device. Indeed, the BTE devices could simply be the same design. That said, this does not require that the BTE device be affirmatively used to meet the method. The method only requires that if this BTE device will utilize, the aforementioned results would be the case. Put another way, this BTE device is a device that can be utilized to test whether or not the method was executed.

In an exemplary embodiment, an orientation of the first implantable component after implantation is at least and/or equal to 30, 35, 40, 45, 50, 55, 60, 65, 70, 75 or 80 degrees or any value or range of values therebetween in 1° increments different than an orientation of the second implantable component after implantation.

Consistent with the concept that in at least some embodiments, the same implant can be utilized for the left side and the right side, the method can include implanting, on a left side of a third human, a third implantable component of a hearing prosthesis including a wide diameter antenna so that the wide diameter antenna is implanted behind or above a pinna of a human, wherein the first and second and third implantable components are the same design. Here a link quality between the antenna system of the third implantable component and the antenna of the external component within the housing of the spine of the BTE device will be effective to communicate data to the antenna system of the third implant so that an effective hearing percept can be evoked in the third human when the external component is worn by the third person in the same way as worn by the first person, albeit on the opposite side of the head, all other things being equal. Again, this BTE device is a control device that can enable a determination as to whether or not the method was executed.

In an exemplary embodiment, the antenna in the housing would be at least partially shadowed by the respective pinnas when the link qualities are achieved. In this regard, the arrangement of FIG. 29 shows the antenna being totally shattered by the pinna. However, if element 810 were moved 1 inch over to the left with respect to the 8.5×11″ sheet of paper upon which that image would be printed upon, no part of element 810 would be shadowed. In an exemplary embodiment, at least and/or equal to 10, 15, 20, 25, 30, 35, 40, 45, 50, 60, 70, 80, 85, 90, 95 or 100% or any value or range of values therebetween in 1% increments of the antenna 810 is shadowed by the pinna with respect to the view of FIG. 29 by geometry and/or by mass. That said in some embodiments, no part of the antenna shadowed by the pinna with respect to the BTE arrangement. With respect to an ITE arrangement, there is no shadowing by the pinna.

This method can further include respectively implanting, on a right side of a third, a fourth, a fifth and a sixth human (or more, implantations up to an nth human, where n can be any integer between 3-100 or any value or range of values therebetween in 1 increments), a respective implantable component of a hearing prosthesis including a wide diameter antenna so that the wide diameter antenna is implanted behind or above a pinna of a third human, wherein the respective implantable components and the first and second implantable components are the same design. Also, the respective orientations of the implantable components after implantation are substantially different from each other, including the orientations of the first and second implantable components. Again, as a control, respective link qualities between the respective antenna systems of the respective implantable components and the antenna of the external component within the housing of the spine of the BTE device will be effective to communicate data to the respective antenna systems of the respective implants so that an effective hearing percept can be evoked in the respective humans when the external component is worn by the respective humans in the same way as worn by the first person, all other things being equal.

It is noted that in an exemplary embodiment, the actions of implantation can be executed within 1, 2, 3, 4, 5, 6, 7, 8, 9, 10, 11, 12, 13, 14, 15, 16, 17, 18, 19 or 10 days or weeks or months. In an exemplary embodiment, the methods can be practiced where there could be an intervening implantation that does not meet the requirements. That is, a method action of implanting five implants meeting the other method actions could be met by implanting three implants consecutively, and then implanting a fourth implant that is outside the method, and then implanting a fifth and sixth implant that meets the method.

An exemplary method further includes respectively secondly implanting, on a left side of a seventh, eighth, ninth and a tenth human (or more, implantations up to an ith human, where i can be any integer between 7-100 or any value or range of values therebetween in 1 increments), a respective second implantable component of a hearing prosthesis including a wide diameter antenna so that the wide diameter antenna is implanted behind or above a pinna of a third human, wherein the respective second implantable components and the first and second implantable components are the same design, respective second orientations of the implantable second components after implantation are substantially different from each other, including the orientations of the first and second implantable components an drespective link qualities between the respective antenna systems of the respective implantable components and the antenna of the external component within the housing of the spine of the BTE device will be effective to communicate data to the antenna system of the respective second implants so that an effective hearing percept can be evoked in the respective humans when the external component is worn by those humans in the same way as worn by the first person, albeit on the opposite side of the head, all other things being equal.

It is also noted that the nomenclature of “first,” “second,” “seventh” etc., is for enumeration and is not used for order unless otherwise stated. Accordingly, for example, the seventh human may be the first, second, third, etc., temporally.

At least some embodiments of the teachings detailed herein can have utilitarian value with respect to achieving a physical implementation of the MI-radio antenna coils of the implant for ipsilateral communication with the external sound processor device (or whatever external device supports the external antenna) that is well-defined and unique. Such can result in, for example, the guarantee of strong or at least statistically significant adequate (i.e., there will be a baseline for the quality of the signal, as the hearing percept evoked by the implant will be based entirely there on in at least some exemplary embodiments where the microphone is located on the outside of the recipient, and thus the teachings detailed herein can meet this baseline in at least 90, 91, 92, 93, 94, 95, 96, 97, 98, 99 or 100% of recipients meeting the 20 to 80 percentile human factors male or female of ages 20 to 80, or in some embodiments, of ages 1 to 80 or 2 to 80 or 3 to 80) to incoming MI implant signals from the external devices (whether such as an ITE, BTE, OTE), avoiding communication interruptions caused by external interference.

In at least some exemplary embodiments of the teachings detailed herein, the arrangements described herein can avoid radio dead zones or close to radio dead zones for signal emanating from the external devices (ITE, BTE, OTE) and/or can avoid antenna positions that are close to the RF power coil (the bigger coil/the wide diameter coil) that result in interface of a statistically significant level.

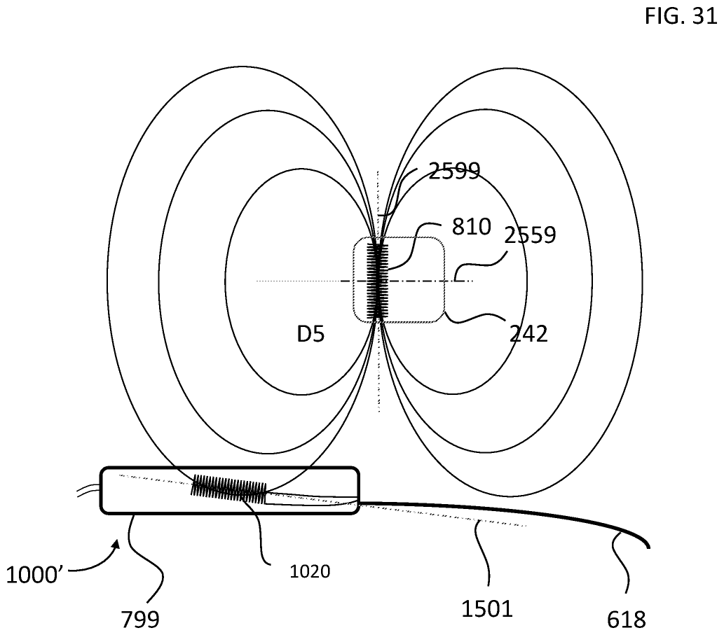

Exemplary embodiments of potential dead zones can be where, with respect to figure for example, the axis 1559 is aligned with the axis 2559. Indeed, dead zones can exist where the magnetic flux is parallel to the axes 1559. In this regard, the magnetic flux of antenna 810, which can be represented as a series of ever-increasing ovals having one side thereof extending through the center of the coil 810. If those ovals are such that they have a tangent component that is parallel with axis 1559, such will result in a dead spot in at least many embodiments. Accordingly, embodiments exist where the placement of the implant are such that the coil antennas of the implant are such that those scenarios resulting in the dead zones do not exist or otherwise are very unlikely to exist. In some exemplary embodiments, the placements of the implant is such that the placement of the antennas results in the axis 1501 being aligned with the tangent component of the aforementioned ovals. FIG. 31 presents an exemplary scenario of use of the external component's MI radio antenna 810 and the magnetic field represented by the ovals, and how the tangent component of the outermost oval is parallel to the axis 1501. Such an arrangement would have good signal strength/good magnetic field strength at the antenna 1020.

In view of the above, it can be seen that in at least some exemplary embodiments, the angle between the axis 1501 and the axis 2599 with respect to the frame of reference of FIG. 26 and/or a frame of reference superimposed upon plane 2772 of FIG. 27, which is a plane that is normal to the direction of gravity and is a plane that is normal to the longitudinal axis of the human (axis 99 represents the longitudinal axis of the human), is at least more than or equal to 5, 6, 7, 8, 9, 10, 11, 12, 13, 14, 15, 20, 25, 30, 35, 40, 45, 50, 55, 60, 65, 70, 75, 80, 85 or 90 degrees or any value or range of values therebetween in 1° increments from orthogonality (i.e., the angle between the two can be 85, 84, 83, 82, degrees, etc.). In an exemplary embodiment, the angles are not orthogonal. In an exemplary embodiment, this can be achieved for the various placements detailed herein for the 20 to 80 percentile human factors engineering male and/or female of the ages detailed herein.

In an exemplary embodiment, providing that the bottom surface of the housing 799 is placed facing the skull, and is implanted in the manner statistically average for a cochlear implant implanted in the United States of America in the year 2019 and/or implanted between the skin and the skull. It is impossible to have a dead zone with respect to the MI radio data transmission antennas when the external component has an antenna 810 aligned such that the longitudinal axis thereof is parallel to the plane that is normal to the longitudinal axes of the human, at least when the respective antennas are within 30 mm of each other.

In an exemplary embodiment, where the first antenna of the implant is a wide diameter coil antenna, the implantable component is configured so that if the first antenna is implanted between skull bone and skin of a human so that the wide diameter extends generally parallel to the skull (and/or the longitudinal axis is normal to the skull surface) and is implanted in the manner statistically average (mean, median and/or mode) for cochlear implantation in the United States of America in the year 2019, if the second antennas of the MI radio link (the implanted antenna and the external antenna being the “second antennas”) are within and/or equal to 20, 25, 30, 35, 40, 45 or 50 mm or any value or range of values therebetween in 1 mm increments) of each other, it is impossible to have a dead zone between the second antenna of the external component and the second antenna of the implantable component if a longitudinal axis of the second antenna of the implantable component is normal to a longitudinal axis of the human.

The physical implementation of the MI-radio antenna coils of the implant for ipsilateral communication with the external sound processor device, in at least some embodiments herein, well-defined. Such can be utilitarian so that the implant can be optimal, or at least statistically adequate, with the BTE/ITE/OTE antenna coil implementation, any one or any two or all three of these being optimized for binaural links. Further, the coils of the implant can be optimized with the OTE antenna coil implementation, the latter residing on top of the RF power coil or proximate thereto and/or within 1, 2, 3, 4, 5, 6, 7, 8, 9 or 10 mm therefrom in some embodiments.

Embodiments can have utilitarian value with respect to avoiding an extra structural antenna lead assembly extending from the housing for the MI antennas. By structural antenna lead assembly, it is meant one that can mechanically support the antenna relative to the housing. This is akin to the lead assemblies that extend to the electrode array or other mechanical stimulating actuator and the lead assemblies that extend to the implanted microphones. These structural lead assemblies not only provide leads for signal communication, but also “hold” the various components together in an assembly. Further by way of example, lead 512 of FIG. 5 is such a structural lead. This as contrasted to the leads of the embodiment of FIG. 1C between the housing and the coil—the silicone body or any other type of biocompatible material such as ceramic or peek material is utilized to hold everything together. This is also contrasted to the leads that extend from the antennas 1020, 1030, etc., which are not structural antenna leads, but instead are solely signal leads. The silicone bodies are other support systems detailed herein are utilized to hold the antennas to the rest of the assembly.

Teachings herein can enable reliable data communication between the external components of the hearing prostheses and the implant along with reliable communication between the various external components in a bilateral arrangement. Such is established, by way of example, by utilizing a separate RF link for data from that which is utilized for power. Embodiments include systems that do not have a combined link and do not include a split link, while other embodiments include these arrangements for redundancy. Indeed, in an exemplary embodiment, upon the failure or otherwise upon the unlikely scenario where the datalink is not sufficiently well-defined, these other links may be utilized as a backstop.

It is noted that any method detailed herein also corresponds to a disclosure of a device and/or system configured to execute one or more or all of the method actions detailed herein. It is further noted that any disclosure of a device and/or system detailed herein corresponds to a method of making and/or using that the device and/or system, including a method of using that device according to the functionality detailed herein. Any functionality disclosed herein also corresponds to a disclosure of a method of executing that functionality, and vice versa.

It is further noted that any disclosure of a device and/or system detailed herein also corresponds to a disclosure of otherwise providing that device and/or system.

Any feature of any embodiment can be combined with any other feature any other embodiment providing that such is enabled. Any feature of any embodiment can be explicitly excluded from utilized nation with any other feature of any embodiment herein providing that the art enable such.

It is noted that in at least some exemplary embodiments, any feature disclosed herein can be utilized in combination with any other feature disclosed herein unless otherwise specified. Accordingly, exemplary embodiments include a medical device including one or more or all of the teachings detailed herein, in any combination.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1-47. (canceled)

48. A method, comprising:
establishing a first transcutaneous link for power transfer using magnetic induction between a first set of closely coupled coil antennas with a clearance less than 20 mm; and
establishing a second transcutaneous link for data communication using magnetic induction between a second set of loosely coupled coil antennas with a clearance greater than 10 mm, wherein
the first and second transcutaneous links are established by the same or respective different external components of a prosthetic hearing implant system and the same implanted component of the prosthetic hearing implant implanted in a human recipient, and
at least one of:
the second transcutaneous link is established using antenna diversity via the implantable component; or
the second transcutaneous link is established using antenna and receiver diversity by the implantable component.

49. The method of claim 48, wherein:
the first transcutaneous link establishes a third link that is a communication link by altering power level or implant load.

50. The method of claim 48, wherein:
the second transcutaneous link is established using antenna and receiver diversity by the implantable component.

51. The method of claim 48, wherein:
the second transcutaneous link is established using antenna diversity via the implantable component.

52. The method of claim 48, wherein:
the second link and the first link are RF frames or bursts within a TDMA scheme.

53. The method of claim 48, wherein:
the second set of antennas comprise narrow diameter coil inductance antennas and the first set of antennas comprise wide diameter coil inductance antennas.

54. The method of claim 48, wherein:
the second link operates at a frequency that is at least half that of the first link or at least twice that of the first link.

55. A method, comprising:
establishing a transcutaneous or subcutaneous data communication link using magnetic induction with an implanted component including magnetic induction radio communication circuitry connected to an implantable antenna arrangement of the implantable component comprising at least two coil antennas, wherein
the method includes selecting one coil antenna of the at least two coil antennas for connection to the magnetic induction radio communication circuitry based on data based on a link quality associated with the selected one coil antenna, which selected antenna is used to establish the communication link.

56. The method of claim 55, wherein:
the action of selecting is executed by comparing a link quality associated with one or more antennas of the at least two coil antennas other than the selected antenna with the link quality associated with the selected antenna.

57. The method of claim 55, wherein:
the data communication link is between the selected one coil antenna and another antenna of another component separate from the implantable component; and
the link qualities respectively correspond to the links between that another antenna and the respective antennas of the at least two coil antennas.

58. The method of claim 57, wherein:
the data communication link is between the selected one coil antenna and another antenna of another component separate from the implantable component; and
the link qualities correspond to the link between respective different antennas of the another component and the respective antennas of the at least two coil antennas.

59. The method of claim 55, further comprising:
electrically decoupling one or more of the at least two coil antennas other than the selected one coil antenna from at least some of the circuitry, based on an evaluation of link quality, and electrically coupling the selected one coil antenna to at least some of the circuitry.

60. The method of claim 55, further comprising:
deactivating a receiver electrically coupled to one or more of the at least two coil antennas other than the selected one coil antenna based on an evaluation of link quality and activating a receiver electrically coupled to the selected one coil antenna, wherein the receivers are part of the circuitry.

61. The method of claim 55, wherein:
the action of selecting is executed after the implantable component has been implanted in a recipient.

62. A method, comprising:
establishing a first transcutaneous link that is a power induction transfer using a first set of antennas; and
establishing a second transcutaneous link for data transfer using a second set of antennas, wherein
the first and second transcutaneous links are established by the same or respective different external components of a prosthetic hearing implant system and the same implanted component of the prosthetic hearing implant implanted in a human recipient, and
the second link utilizes induction to communicate the data, wherein
the prosthetic hearing implant implanted in the human recipient includes at least two coil antennas of which an antenna of the first set and an antenna of the second set are apart.

63. The method of claim 62, wherein:
the data link and the power link are RF frames or bursts within a TDMA scheme.

64. The method of claim 62, wherein:
the second set of antennas is established by a first antenna from an external (non-implantable) component close to or in an ear canal of the recipient and the antenna arrangement of the second set of antennas implanted in the recipient; and
the method further comprises, after establishing the second link, establishing a third transcutaneous data link using a third antenna from an external (non-implantable) component close to or in the ear canal of the recipient and the antenna arrangement of the implantable component of the second set of antennas.

65. The method of claim 62, wherein:
the second set of antennas comprise narrow diameter coil inductance antennas and the first set of antennas comprise wide diameter coil inductance antennas.

66. The method of claim 62, wherein:
the first power link is an inductance link; and
the second data link is an MI radio link.

67. The method of claim 62, wherein:
the second link operates at a frequency that is at least half that of the first link or at least twice that of the first link.

68-82. (canceled)
